(19) United States
(12) Patent Application Publication
(10) Pub. No.: US 2022/0340658 A1
(43) Pub. Date: Oct. 27, 2022

(54) NEUTRALIZING ANTI-SARS-CoV-2 ANTIBODIES AND USE THEREOF

(71) Applicant: Washington University, St. Louis, MO (US)

(72) Inventors: Michael Diamond, St. Louis, MO (US); Laura VanBlargan, St. Louis, MO (US); Daved Fremont, St. Louis, MO (US); Lucas Adams, St. Louis, MO (US)

(21) Appl. No.: 17/729,820

(22) Filed: Apr. 26, 2022

Related U.S. Application Data
(60) Provisional application No. 63/179,978, filed on Apr. 26, 2021.

(51) Int. Cl. C07K 16/28; A61P 11/00; A61P 31/14

(52) U.S. Cl. CPC C07K 16/283; A61P 11/00; A61P 31/14; C07K 2317/70; C07K 2317/92; C07K 2317/24

(57) ABSTRACT

The present invention relates to antibodies or antigen-binding fragments that are useful for treating coronavirus infections (e.g., COVID-19 caused by SARS-CoV-2). The present invention also relates to various pharmaceutical compositions and methods of treating coronavirus using the antibodies or antigen-binding fragments.

### CROSS REFERENCE TO RELATED APPLICATIONS

This application claims the benefit of U.S. Provisional Application No. 63/179,978, filed Apr. 26, 2021 the disclosure of which is hereby incorporated by reference in its entirety.

### FIELD OF THE TECHNOLOGY

The present disclsoure relates to antibodies or antigen-binding fragments that are useful for treating infections caused by coronaviruses (e.g., SARS-CoV-2). The present invention also relates to various pharmaceutical compositions and methods of treating coronavirus infections (e.g., COVID-19) using the antibodies or antigen-binding fragments.

### REFERENCE TO SEQUENCE LISTING

This application contains a Sequence Listing that has been submitted in ASCII format via EFS-Web and is hereby incorporated by reference in its entirety. The ASCII copy, created on Apr. 26, 2022, is named Untitled_ST25.txt, and is 24,576 bytes in size.

### BACKGROUND

Several members of the family Coronaviridae typically affect the respiratory tract of mammals, including humans, and usually cause mild respiratory disease. In the past two decades, however, two highly pathogenic coronaviruses (CoVs), including severe acute respiratory syndrome coronavirus (SARS-CoV) and Middle East respiratory syndrome coronavirus (MERS-CoV), have crossed the species barrier and led to global epidemics with high morbidity and mortality. SARS-CoV first appeared in 2002 in the Guangdong province of China and then quickly spread as a global epidemic in more than 30 countries, infecting 8,098 people and causing 774 deaths. In 2012, MERS-CoV emerged in the Arabian Peninsula, and its subsequent spread to 27 countries was associated with 2,494 confirmed cases and 858 deaths. In December 2019, the third highly pathogenic human coronavirus (HCoV), 2019 novel coronavirus (2019-nCoV), as denoted by the World Health Organization (WHO), was discovered in Wuhan, Hubei province of China. 2019-nCoV, with 79.5 and 96% sequence identity to SARS-CoV and a bat coronavirus, SL-CoV-RaTG13, respectively, was then renamed SARS-CoV-2 by the Coronaviridae Study Group (CSG) of the International Committee on Taxonomy of Viruses (ICTV). Compared to SARS-CoV and MERS-CoV, SARS-CoV-2 appears to be more readily transmitted from human-to-human, spreading to multiple continents and leading to the WHO declaration of a global pandemic on Mar. 11, 2020.

There is a need for compostions and methods for treating this virulent infection. For example, specific antibodies that can target and neutralize SARS-CoV-2 (or other related SARS or MERS coronaviruses) are useful to treat or prevent active COVID-19 infections and are provided herein.

### BRIEF DESCRIPTION OF THE FIGURES

The application file contains at least one photograph executed in color. Copies of this patent application publication with color photographs will be provided by the Office upon request and payment of the necessary fee.

FIG. 1 shows a Panel of anti-SARS-CoV-2 mAbs. Hybridoma supernatants from the panel of anti-SARS-CoV-2 murine mAbs were assayed for neutralization of SARS-CoV-2 by FRNT, cross-reactivity to SARS-CoV-1 spike protein, and ability to inhibit SARS-CoV-2 spike protein binding to hACE2 or a panel of reference human mAbs through competition ELISA. MAbs are grouped by reference mAb competition properties. Data represent the mean (or geometric mean for EC50 values) from two to four independent experiments. Hybridomas were produced from splenocytes of mice that received three immunizations (once with the RBD and then twice with Spike) prior to a final pre-fusion boost with either RBD or Spike, as indicated in the ‘Final Boost’ column.

FIG. 2A, FIG. 2B, FIG. 2C, FIG. 2D, FIG. 2E, FIG. 2F, FIG. 2G and FIG. 2H show neutralization by anti-SARS-CoV-2 mAbs. FIG. 2A and FIG. 2B show anti-SARS-CoV-2 mAbs were assayed for neutralization by FRNT against SARS-CoV-2 using Vero E6 cells. FIG. 2A shows representative dose response curves are shown. FIG. 2B shows mean EC50 values; data are from three to four experiments. FIG. 2C and FIG. 2D show anti-SARS-CoV-2 mAbs were assayed for pre- or post-attachment neutralization of SARS-CoV-2 using Vero E6 cells. FIG. 2C shows fold change in EC50 values for post-attachment over pre-attachment neutralization. Error bars represent standard error of the mean (SEM) from four experiments. FIG. 2D shows representative dose response curves are shown. FIG. 2E shows anti-SARS-CoV-2 mAbs were assayed for pre- or post-attachment inhibition on Vero-TMPRSS2-ACE2 cells. Dose response curves are shown. Data are representative of three experiments. FIG. 2F and FIG. 2G show anti-SARS-CoV-2 mAbs were assayed for attachment inhibition of SARS-CoV-2 to Vero E6, Vero-TMPRSS2, or Vero-TMPRSS2-ACE2 (FIG. 2F) or Calu-3 (FIG. 2G) cells. Data are from three (FIG. 2F) to six (FIG. 2G) experiments. FIG. 2H shows anti-SARS-CoV-2 mAbs were assayed for inhibition of virus internalization in Vero E6 cells. Data are from four experiments. FIG. 2C. ANOVA with Sidak's post-test comparing pre- vs. post-attachment EC50 values for each mAb; FIG. 2F-FIG. 2H. One-way ANOVA with Dunnett's post-test compared mAb treatment to isotype control mAb treatment. ns, not significant; *p<0.05; **p<0.01; ***p<0.001, ****p<0.0001.

FIG. 3A, FIG. 3B, FIG. 3C, FIG. 3D, FIG. 3E and FIG. 3F show epitopes recognized by anti-SARS-CoV-2 mAbs. mAbs were tested for neutralization potency against a panel of VSV-eGFP-SARS-COV-2-S neutralization escape mutants. FIG. 3A “+” symbol indicates resistance to neutralization when a mutation at the indicated residue number is present. FIG. 3A-3F show residues from (FIG. 3A) are highlighted on the RBD structure (PDB 6M0J) in red, orange, green, or cyan for mAbs from group A, B, C, or D, respectively, and indicated. Residues that engage hACE2 are highlighted in tan.

FIG. 4A, FIG. 4B, FIG. 4C, FIG. 4D, FIG. 4E, FIG. 4F, FIG. 4G, FIG. 4H, and FIG. 4I show Anti-SARS-CoV-2 mAbs protect against SARS-CoV-2 infection in vivo. FIG. 4A-FIG. 4G show K18-hACE2 transgenic mice were passively administered 100 μg (5 mg/kg) of the indicated mAb by intraperitoneal injection 24 h prior to intranasal inoculation with 10³ FFU of SARS-CoV-2 WA1/2020. FIG. 4A-FIG. 4D Mice were monitored for weight change for 7 days following viral infection. Mean weight change is shown. Error bars represent SEM. (FIG. 4E-FIG. 4F) At 7 dpi, nasal washes (FIG. 4E), and lungs (FIG. 4F) were collected, and viral RNA levels were determined. Median levels are shown; top dotted line indicates median viral load of control mAb-treated mice; bottom dotted line represents the limit of detection (LOD) of the assay. (FIG. 4H) A subset of the lungs from (FIG. 4F) were assessed for infectious viral burden by plaque assay. Median PFU/mL is shown. Dotted line indicates the LOD. (FIG. 4A-FIG. 4F) Data for each mAb are from two experiments; WEEV-204 (isotype control): n=12; all other mAbs: n=5-6 per group. (FIG. 4H-FIG. 4I) K18-hACE2 transgenic mice were passively given 200 μg (10 mg/kg) of the indicated mAb by intraperitoneal injection 24 h after intranasal inoculation with 103 PFU of SARS-CoV-2 WA1/2020. Data are from two or three experiments; WEEV-204 (isotype control): n=10; SARS2-02 and SARS2-38: n=6 per group. (FIG. 4H) Mean weight change is shown. Error bars represent SEM. (FIG. 4I) At 7 dpi, lung, nasal washes, heart, and brain were collected and viral RNA levels were determined. (FIG. 4A-FIG. 4D and FIG. 4H) One-way ANOVA with Dunnett's post-test of area under the curve. ns, not significant; ****p<0.0001. (FIG. 4E, FIG. 4F, and FIG. 4I) Kruskal-Wallis with Dunn's post-test: ns, not significant, *p<0.05, ** p<0.01, ***p<0.001, ****p<0.0001.

FIG. 5A, FIG. 5B, FIG. 5C, FIG. 5D, FIG. 5E, FIG. 5F, FIG. 5G, FIG. 5H, FIG. 5I, FIG. 5J and FIG. 5K show neutralization of variants of concern by anti-SARS-CoV-2 mAbs. FIG. 5A shows Variants of concern (VOC) and their mutations in spike. SARS2-2 (FIG. 5A-FIG. 5B) and SARS2-38 (FIG. 5D-FIG. 5E) were tested for neutralization of the indicated variants by FRNT. FIG. 5B and FIG. 5D show representative dose response curves are shown. FIG. 5C and FIG. 5E show mean EC50 values are shown; data are from three to five experiments. FIG. 5F-FIG. 5G show representative dose response curves of SARS2-02 and SARS2-38 neutralization of VSV-eGFP-SARS-CoV-2-S and the indicated neutralization-resistant mutants. Data is from of one of two experiments. (FIG. 5H-FIG. 5I) K18-hACE2 mice were administered 100 μg (5 mg/kg) of the indicated mAb by intraperitoneal injection 24 h prior to intranasal inoculation with 10³ FFU of SARS-CoV-2 Wash-B.1.351. (FIG. 5H) Mean weight change is shown. Error bars represent SEM. (FIG. 5I) At 6 dpi, the indicated tissues were collected, and viral RNA levels were determined. Data are from two experiments; WEEV-204 (isotype control) and SARS2-38: n=7; SARS2-02: n=6. (FIG. 5J-FIG. 5K) K18-hACE2 mice were inoculated with 10³ FFU of SARS-CoV-2 Wash-B.1.351, and 24 h later they were administered 200 μg (10 mg/kg) of the indicated mAb. (FIG. 5J) Mean weight change is shown. Error bars represent SEM. (FIG. 5K) At 6 dpi, the indicated tissues were collected, and viral RNA levels were determined. Data are from two experiments; hWNV-E16 and hSARS2-02: n=6; hSARS2-38: n=8). (FIG. 5H and FIG. 5J) One-way ANOVA with Dunnett's post-test of area under the curve. **p<0.01; ****p<0.0001. (FIG. 5I and FIG. 5K) Kruskal-Wallis with Dunn's post-test: ns, not significant, *p<0.05, **p<0.01, ***p<0.001, ****p<0.0001.

FIG. 6A, FIG. 6B and FIG. 6C show SARS2-38 targets a conserved portion of the RBM with extensive light chain contact. FIG. 6A Left panel. Density map of SARS2-38 Fv bound to trimeric SARS-CoV-2 spike protein with all RBDs in the down position. The spike monomer bound by SARS2-38 is shown in yellow with the rest of the trimer colored gray. The SARS2-38 heavy chain is shown in royal blue, and the light chain in cyan. Middle panel. Focused density map of the Fv/RBD complex encompassing a refined atomic model. The RBD is shown in yellow. The SARS2-38 heavy and light chains are colored royal blue and cyan, respectively. Right panel. Complementarity-determining regions (CDRs) of SARS2-38 overlay a surface rendering of the RBD. CDRs from the heavy and light chains are colored royal blue and cyan, respectively, with the RBD colored yellow. ACE2-binding residues of the receptor binding motif (RBM) are outlined in green. (FIG. 6B) Left panel: a ribbon diagram of the RBD and SARS2-38 CDRs with escape mutations and variants of concern (VOCs) noted in purple and red, respectively. The RBD is otherwise colored yellow, with a gray glycan linked to N343. CDRs of the SARS2-38 heavy and light chain are colored royal blue and cyan, respectively. Right panel: surface renderings of RBD colored according to conservation of surface residues (blue=conserved, red=variable). Escape mutations and VOCs are noted in purple and red, respectively. The SARS2-38 epitope is outlined in navy. (FIG. 6C) Multiple sequence alignment of RBD (residues 333-518) from WA1/2020, SARS-CoV-2 VOCs, SARS-CoV, and MERS-CoV, with the binding footprint of SARS2-38 boxed in blue. Mutations within SARS-CoV-2 VOCs are highlighted in red, and SARS2-38 escape mutation contacts are marked with purple triangles. Secondary structure annotation is displayed above the alignment in yellow with ACE2 contacts designated by green triangles. Divergent residues within SARS-CoV and MERS-CoV (relative to WA1/2020) are highlighted in gray. (SEQ ID NOs: 9-17)

FIG. 7A and FIG. 7B show similarity of SARS2-38 epitope to other mAbs. FIG. 7A show Structural comparison of SARS2-38 to mAbs targeting a similar region of the RBD. (FIG. 7B) Multiple sequence alignment of the SARS-CoV-2 RBD (residues 333-518) with mAb binding footprints as determined by qtPISA analysis. For SARS2-38, heavy chain, light chain, and shared contacts are shown in blue, cyan, and dark blue, respectively. For 2H04, heavy chain, light chain, and shared contacts are shown in orange, pale orange, and dark orange, respectively. For REGN10987, heavy chain, light chain, and shared contacts are shown in green, pale green, and dark green, respectively. For S309, heavy chain, light chain, and shared contacts are shown in magenta, pale purple, and purple, respectively. For COV2-2130, heavy chain, light chain, and shared contacts are shown in red, pale red, and brick red, respectively. Secondary structure annotation is displayed above the alignment in yellow, with ACE2 contacts designated by green triangles. VOC substitutions are designated below the alignment by red triangles. (SEQ ID NO: 8)

### DETAILED DESCRIPTION

The present disclosure is based, at least in part, on the discovery of various antibodies and antigen-binding fragments thereof that show specificity to coronaviruses. Antibodies and antigen-binding fragments thereof described herein can neutralize the virus in vitro and in vivo. In particular, the present disclosure identifies a conserved epitope proximal to the receptor binding motif within the SARS-CoV-2 spike protein that when bound by an inhibitory antibody potently neutralizes many SARS-CoV-2 variants of concern. Thus, the present disclosure provides compostions and methods treatment with or induction of inhibitory antibodies that bind conserved spike epitopes and limit the loss of potency of therapies or vaccines against emerging SARS-CoV-2 variants.

Disclosed herein are compositions, methods, and treatment plans for treating an individual who is at risk of having a respiratory viral infection, has mild symptoms of a respiratory viral infection, or has severe symptoms of a respiratory viral infection. A composition of the present disclosure comprising an antibody and/or antigen-binding fragment disclosed herein may be used to treat, prevent, or reduce the infectivity of a respiratory viral infection. A treatment plan may comprise administering a composition (e.g., a composition comprising an antibody and/or antigen-binding fragment of the disclosure) to an individual at risk of having a viral infection or who has a viral infection, thereby preventing or treating the viral infection. In some embodiments, a viral infection may be prevented by reducing the amount of virus capable of binding to a host cell or tissue. For example, a composition of the present disclosure may comprise an antibody and/or antigen-binding fragment of the disclsoure and a viral infection may be prevented by disrupting interactions between a viral surface proteins and host cell proteins that activate or enhance insertion of the viral genetic material into the host cell. For example, interactions between a SARS-CoV-2 spike protein, and a host cell ACE-2 receptor.

### I. Definitions

The term “a” or “an” entity refers to one or more of that entity; for example, a “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone).

Unless defined otherwise, technical and scientific terms used herein have the same meaning as commonly understood by one of ordinary skill in the art to which this disclosure is related.

Where applicable, units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. Nucleic acid sequences are written from 5′ to 3′, left to right.

The headings provided herein are not limitations of the various aspects and embodiments of the disclosure, which can be had by reference to the specification as a whole.

Terms defined immediately below are more fully defined by reference to the specification in its entirety.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of amino acid monomers linearly linked by peptide bonds (also known as amide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, hydrophobic interactions, etc., to produce, e.g., a multimeric protein.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or might be at any time, determined or interpreted by a judge or an administrative or judicial body to be, “naturally-occurring.”

Other polypeptides disclosed herein are fragments, derivatives, analogs, or variants of the foregoing polypeptides, and any combination thereof. The terms “fragment,” “variant,” “derivative” and “analog” when referring to polypeptide subunit or multimeric protein as disclosed herein can include any polypeptide or protein that retain at least some of the activities of the complete polypeptide or protein, but which is structurally different. Fragments of polypeptides include, for example, proteolytic fragments, as well as deletion fragments. Variants include fragments as described above, and also polypeptides with altered amino acid sequences due to amino acid substitutions, deletions, or insertions. Variants can occur spontaneously or be intentionally constructed. Intentionally constructed variants can be produced using art-known mutagenesis techniques. Variant polypeptides can comprise conservative or non-conservative amino acid substitutions, insertions, and/or deletions. Derivatives are polypeptides that have been altered so as to exhibit additional features not found on the native polypeptide, such as increased resistance to proteolytic degradation. Examples include fusion proteins. Variant polypeptides can also be referred to herein as “polypeptide analogs.” As used herein a “derivative” also refers to a subject polypeptide having one or more amino acids chemically derivatized by reaction of a functional side group. Also included as “derivatives” are those peptides that contain one or more standard or synthetic amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate protein activity are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:.412-417 (1997)).

As used herein, the term “binding molecule” refers in its broadest sense to a molecule that specifically binds an antigenic determinant. As described further herein, a binding molecule can comprise one of more “binding domains.” As used herein, a “binding domain” is a two- or three-dimensional polypeptide structure that cans specifically bind a given antigenic determinant, or epitope. A non-limiting example of a binding molecule is an antibody or fragment thereof that comprises a binding domain that specifically binds an antigenic determinant or epitope. Another example of a binding molecule is a bispecific antibody comprising a first binding domain binding to a first epitope, and a second binding domain binding to a second epitope.

Disclosed herein are certain binding molecules, or antigen-binding fragments, variants and/or derivatives thereof. Unless specifically referring to full-sized antibodies such as naturally-occurring antibodies, the term “binding molecule” encompasses full-sized antibodies as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

By “specifically binds,” it is meant that a binding molecule, e.g., an antibody or antigen-binding fragment thereof binds to an epitope via its antigen binding domain, and that the binding entails some recognition between the antigen binding domain and the epitope. According to this definition, a binding molecule is said to “specifically bind” to an epitope when it binds to that epitope, via its antigen-binding domain binds more readily than it would bind to a random, unrelated epitope.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition, or disorder or those in which the disease, condition or disorder is to be prevented.

The term “pharmaceutical composition” refers to a preparation that is in such form as to permit the biological activity of the active ingredient to be effective and does not contain components that are unacceptably toxic to a subject to which the composition would be administered. Such composition can be sterile.

An “effective amount” as disclosed herein is an amount sufficient to carry out a specifically stated purpose. An “effective amount” can be determined empirically and in a routine manner, in relation to the stated purpose.

Coronavirus is a family of positive-sense, single-stranded RNA viruses that are known to cause severe respiratory illness. Viruses currently known to infect human from the coronavirus family are from the alphacoronavirus and betacoronavirus genera. Additionally, it is believed that the gammacoronavirus and deltacoronavirus genera may infect humans in the future. Non-limiting examples of betacoronaviruses include Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIVI-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV).

The viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene included in the 5′-portion of the genome, and structural genes included in the 3′-portion of the genome.

Coronavirus Spike (S) protein: A class I fusion glycoprotein initially synthesized as a precursor protein. Individual precursor S polypeptides form a homotrimer and undergo glycosylation within the Golgi apparatus as well as processing to remove the signal peptide, and cleavage by a cellular protease to generate separate SI and S2 polypeptide chains, which remain associated as S1/S2 protomers within the homotrimer and is therefore a trimer of heterodimers. The S1 subunit is distal to the virus membrane and contains the receptor-binding domain (RBD) that mediates virus attachment to its host receptor. The S2 subunit contains fusion protein machinery, such as the fusion peptide, two heptadrepeat sequences (HR1 and HR2) and a central helix typical of fusion glycoproteins, a transmembrane domain, and the cytosolic tail domain.

Coronavirus Spike (S) protein prefusion conformation is a structural conformation adopted by the ectodomain of the coronavirus S protein following processing into a mature coronavirus S protein in the secretory system, and prior to triggering of the fusogenic event that leads to transition of coronavirus S to the post fusion conformation. The three-dimensional structure of an exemplary coronavirus S protein (HKU1-CoV) in a prefusion conformation is disclosed herein and provided in Kirchdoerfer et al., “Prefusion structure of a human coronavirus spike protein,” Nature, 531: 118-121, 2016 (incorporated by reference herein).

A coronavirus S ectodomain trimer “stabilized in a prefusion conformation” comprises one or more amino acid substitutions, deletions, or insertions compared to a native coronavirus S sequence that provide for increased retention of the prefusion conformation compared to coronavirus S ectodomain trimers formed from a corresponding native coronavirus S sequence. The “stabilization” of the prefusion conformation by the one or more amino acid substitutions, deletions, or insertions can be, for example, energetic stabilization (for example, reducing the energy of the prefusion conformation relative to the post fusion open conformation) and/or kinetic stabilization (for example, reducing the rate of transition from the prefusion conformation to the post fusion conformation). Additionally, stabilization of the coronavirus S ectodomain trimer in the prefusion conformation can include an increase in resistance to denaturation compared to a corresponding native coronavirus S sequence. Methods of determining if a coronavirus S ectodomain trimer is in the prefusion conformation are provided herein, and include (but are not limited to) negative-stain electron microscopy and antibody binding assays using a prefusion-conformation-specific antibody.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

In one example, a desired response is to inhibit or reduce or prevent CoV (such as SARS-CoV-2) infection. The CoV infection does not need to be completely eliminated or reduced or prevented for the method to be effective. For example, administration of an effective amount of the immunogen can induce an immune response that decreases the CoV infection (for example, as measured by infection of cells, or by number or percentage of subjects infected by the CoV) by a desired amount, for example by at least 50%, at least 60%, at least 70%, at least 80%, at least 90%, at least 95%, at least 98%, or even at least 100% (elimination or prevention of detectable CoV infection), as compared to a suitable control. Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on coronavirus S ectodomain, such as a SARS-CoV S ectodomain. Epitopes can be formed both from contiguous amino acids or noncontiguous amino acids juxtaposed by tertiary folding of a protein.

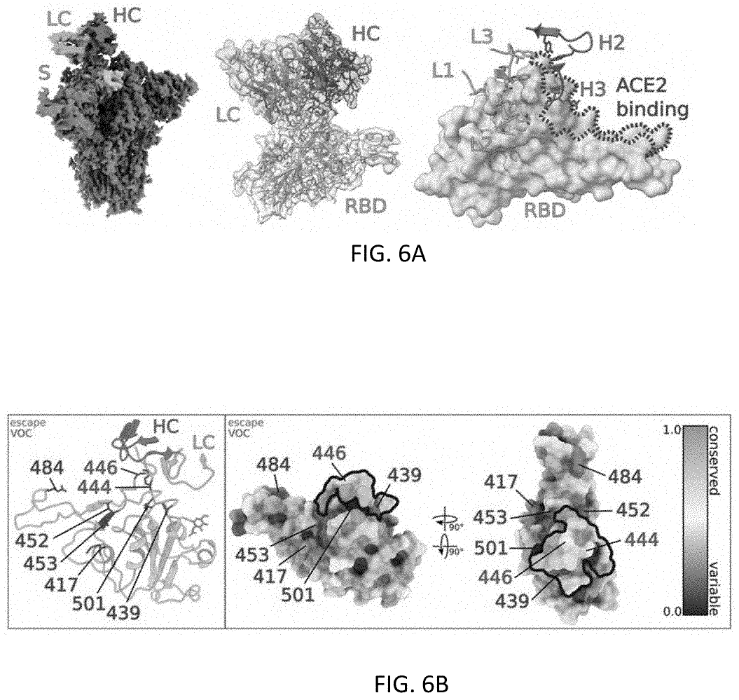

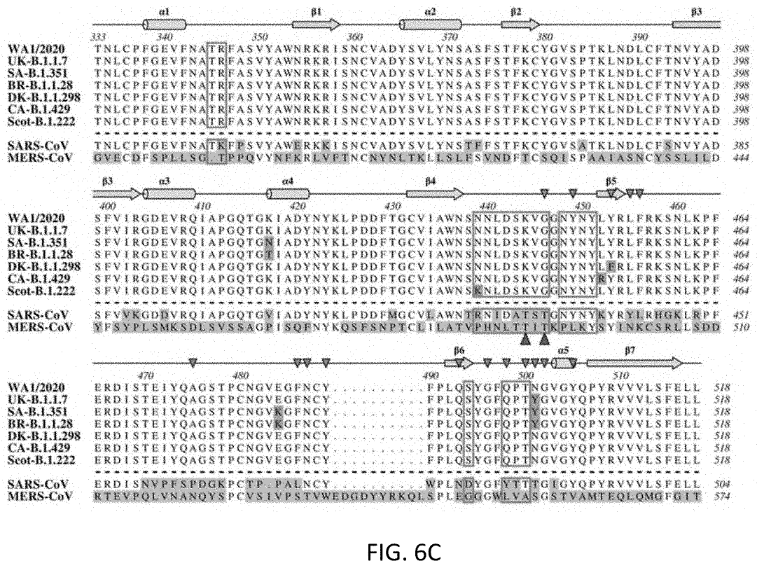

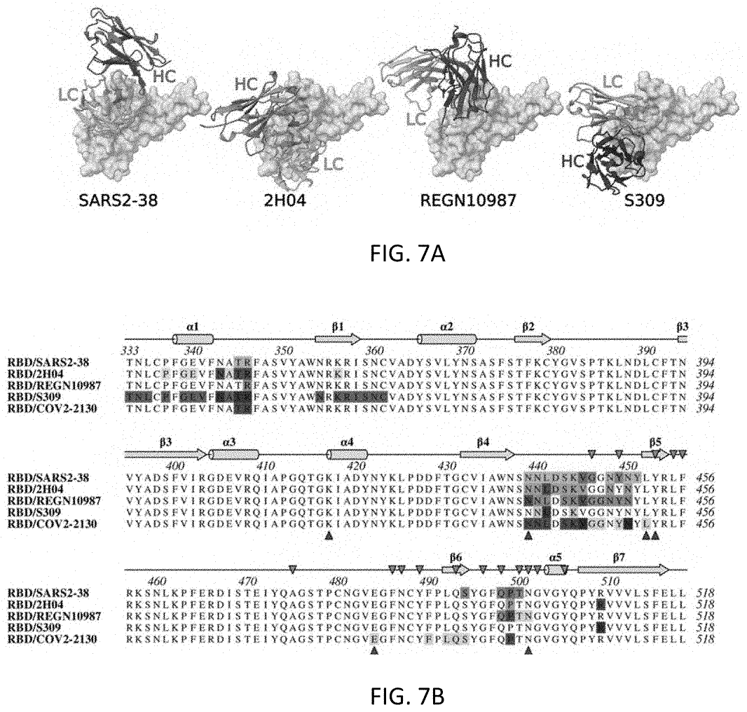

The term “CoV-S”, also called “S” or “S protein” refers to the spike protein of a coronavirus, and can refer to specific S proteins such as SARS-CoV-2-S, MERS-CoV S, and SARS-CoV S. The SARS-CoV-2-Spike protein is a 1273 amino acid type I membrane glycoprotein which assembles into trimers that constitute the spikes or peplomers on the surface of the enveloped coronavirus particle. The protein has two essential functions, host receptor binding and membrane fusion, which are attributed to the N-terminal (S1) and C-terminal (S2) halves of the S protein. CoV-S binds to its cognate receptor via a receptor binding domain (RBD) present in the S1 subunit. The amino acid sequence of the RBD with reference to the full-length SARS-CoV-2 spike protein is exemplified in FIG. 6 and FIG. 7. The term “CoV-S” includes protein variants of CoV spike protein isolated from different CoV isolates as well as recombinant CoV spike protein or a fragment thereof. The term also encompasses CoV spike protein or a fragment thereof coupled to, for example, a histidine tag, mouse or human Fc, or a signal sequence such as ROR1.

The term “antibody,” as used herein, is used in the broadest sense and encompasses various antibody and antibody-like structures, including but not limited to full-length monoclonal, polyclonal, and multispecific (e.g., bispecific, trispecific, etc.) antibodies, as well as heavy chain antibodies and antibody fragments provided they exhibit the desired antigen-binding activity. The domain(s) of an antibody that is involved in binding an antigen is referred to as a “variable region” or “variable domain,” and is described in further detail below. A single variable domain may be sufficient to confer antigen-binding specificity. Preferably, but not necessarily, antibodies useful in the discovery are produced recombinantly. Antibodies may or may not be glycosylated, though glycosylated antibodies may be preferred. An “isolated” antibody is one which has been separated from a component of its natural environment. In some embodiments, an antibody is purified to greater than 95% or 99% purity as determined by methods known in the art.

In addition to antibodies described herein, it may be possible to design an antibody mimetic or an aptamer using methods known in the art that functions substantially the same as an antibody of the invention. An “antibody mimetic” refers to a polypeptide or a protein that can specifically bind to an antigen but is not structurally related to an antibody. Antibody mimetics have a mass of about 3 kDa to about 20 kDa. Non-limiting examples of antibody mimetics are affibody molecules, affilins, affimers, alphabodies, anticalins, avimers, DARPins, and monobodies. Aptamers are a class of small nucleic acid ligands that are composed of RNA or single-stranded DNA oligonucleotides and have high specificity and affinity for their targets. Aptamers interact with and bind to their targets through structural recognition, a process similar to that of an antigen-antibody reaction. Aptamers have a lower molecular weight than antibodies, typically about 8-25 kDa.

The terms “full length antibody” and “intact antibody” may be used interchangeably, and refer to an antibody having a structure substantially similar to a native antibody structure or having heavy chains that contain an Fc region as defined herein. The basic structural unit of a native antibody comprises a tetramer. Each tetramer is composed of two identical pairs of polypeptide chains, each pair having one “light” chain (about 25 kDa) and one “heavy” chain (about 50-70 kDa). Light chains are classified as gamma, mu, alpha, and lambda. Heavy chains are classified as gamma, mu, alpha, delta, or epsilon, and define the antibody's isotype as IgG, IgM, IgA, IgD and IgE, respectively. The amino-terminal portion of each light and heavy chain includes a variable region of about 100 to 110 or more amino acid sequences primarily responsible for antigen recognition (VL and VH, respectively). The carboxy-terminal portion of each chain defines a constant region primarily responsible for effector function. Within light and heavy chains, the variable and constant regions are joined by a “J” region of about 12 or more amino acid sequences, with the heavy chain also including a “D” region of about 10 more amino acid sequences. Intact antibodies are properly cross-linked via disulfide bonds, as is known in the art.

The variable domains of the heavy chain and light chain of an antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Framework region” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence: FR1-HVR1-FR2-HVR2-FR3-HVR3-FR4. The FR domains of a heavy chain and a light chain may differ, as is known in the art.

The term “hypervariable region” or “HVR” as used herein refers to each of the regions of a variable domain which are hypervariable in sequence (also commonly referred to as “complementarity determining regions” or “CDR”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). As used herein, “an HVR derived from a variable region” refers to an HVR that has no more than two amino acid substitutions, as compared to the corresponding HVR from the original variable region. Exemplary HVRs herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and (d) combinations of (a), (b), and/or (c), as defined below for various antibodies of this disclosure. Unless otherwise indicated, HVR residues and other residues in the variable domain (e.g., FR residues) are numbered herein according to Kabat et al., supra.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “variant Fc region” comprises an amino acid sequence that can differ from that of a native Fc region by virtue of one or more amino acid substitution(s) and/or by virtue of a modified glycosylation pattern, as compared to a native Fc region or to the Fc region of a parent polypeptide. In an example, a variant Fc region can have from about one to about ten amino acid substitutions, or from about one to about five amino acid substitutions in a native sequence Fc region or in the Fc region of the parent polypeptide. The variant Fc region herein may possess at least about 80% homology, at least about 90% homology, or at least about 95% homology with a native sequence Fc region and/or with an Fc region of a parent polypeptide.

An “antibody fragment” refers to a molecule other than an intact antibody that comprises a portion of an intact antibody that binds the antigen to which the intact antibody binds. Non-limiting examples of antibody fragments include but are not limited to Fv, Fab, Fab′, Fab′-SH, F(ab′)₂; single-chain forms of antibodies and higher order variants thereof; single-domain antibodies, and multispecific antibodies formed from antibody fragments.

Single-chain forms of antibodies, and their higher order forms, may include, but are not limited to, single-domain antibodies, single chain variant fragments (scFvs), divalent scFvs (di-scFvs), trivalent scFvs (tri-scFvs), tetravalent scFvs (tetra-scFvs), diabodies, and triabodies and tetrabodies. ScFv's are comprised of heavy and light chain variable regions connected by a linker. In most instances, but not all, the linker may be a peptide. A linker peptide is preferably from about 5 to 30 amino acids in length, or from about 10 to 25 amino acids in length. Typically, the linker allows for stabilization of the variable domains without interfering with the proper folding and creation of an active binding site. In preferred embodiments, a linker peptide is rich in glycine, as well as serine or threonine. ScFvs can be used to facilitate phage display or can be used for flow cytometry, immunohistochemistry, or as targeting domains. Methods of making and using scFvs are known in the art. ScFvs may also be conjugated to a human constant domain (e.g. a heavy constant domain is derived from an IgG domain, such as IgG1, IgG2, IgG3, or IgG4, or a heavy chain constant domain derived from IgA, IgM, or IgE). Diabodies, triabodies, and tetrabodies and higher order variants are typically created by varying the length of the linker peptide from zero to several amino acids. Alternatively, it is also well known in the art that multivalent binding antibody variants can be generated using self-assembling units linked to the variable domain.

An antibody of the disclosure may be a Dual-affinity Re-targeting Antibody (DART). The DART format is based on the diabody format that separates cognate variable domains of heavy and light chains of the 2 antigen binding specificities on 2 separate polypeptide chains. Whereas the 2 polypeptide chains associate noncovalently in the diabody format, the DART format provides additional stabilization through a C-terminal disulfide bridge. DARTs can be produced in high quantity and quality and reveal exceptional stability in both formulation buffer and human serum.

A “single-domain antibody” refers to an antibody fragment consisting of a single, monomeric variable antibody domain.

Multispecific antibodies include bi-specific antibodies, tri-specific, or antibodies of four or more specificities. Multispecific antibodies may be created by combining the heavy and light chains of one antibody with the heavy and light chains of one or more other antibodies. These chains can be covalently linked.

“Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

A “heavy chain antibody” refers to an antibody that consists of two heavy chains. A heavy chain antibody may be an IgG-like antibody from camels, llamas, alpacas, sharks, etc., or an IgNAR from a cartiliaginous fish.

A “humanized antibody” refers to a non-human antibody that has been modified to reduce the risk of the non-human antibody eliciting an immune response in humans following administration but retains similar binding specificity and affinity as the starting non-human antibody. A humanized antibody binds to the same or similar epitope as the non-human antibody. The term “humanized antibody” includes an antibody that is composed partially or fully of amino acid sequences derived from a human antibody germ line by altering the sequence of an antibody having non-human hypervariable regions (“HVR”). The simplest such alteration may consist simply of substituting the constant region of a human antibody for the murine constant region, thus resulting in a human/murine chimera which may have sufficiently low immunogenicity to be acceptable for pharmaceutical use. Preferably, the variable region of the antibody is also humanized by techniques that are by now well known in the art. For example, the framework regions of a variable region can be substituted by the corresponding human framework regions, while retaining one, several, or all six non-human HVRs. Some framework residues can be substituted with corresponding residues from a non-human VL domain or VH domain (e.g., the non-human antibody from which the HVR residues are derived), e.g., to restore or improve specificity or affinity of the humanized antibody. Substantially human framework regions have at least about 75% homology with a known human framework sequence (i.e. at least about 75%, at least about 80%, at least about 85%, at least about 90%, at least about 95%, or at least about 99% sequence identity). HVRs may also be randomly mutated such that binding activity and affinity for the antigen is maintained or enhanced in the context of fully human germline framework regions or framework regions that are substantially human. As mentioned above, it is sufficient for use in the methods of this discovery to employ an antibody fragment. Further, as used herein, the term “humanized antibody” refers to an antibody comprising a substantially human framework region, at least one HVR from a nonhuman antibody, and in which any constant region present is substantially human. Substantially human constant regions have at least about 90% with a known human constant sequence (i.e. about 90%, about 95%, or about 99% sequence identity). Hence, all parts of a humanized antibody, except possibly the HVRs, are substantially identical to corresponding pairs of one or more germline human immunoglobulin sequences.

If desired, the design of humanized immunoglobulins may be carried out as follows, or using similar methods familiar to those with skill in the art (for example, see Almagro, et al. Front. Biosci. 2008, 13(5):1619-33). A murine antibody variable region is aligned to the most similar human germ line sequences (e.g. by using BLAST or similar algorithm). The CDR residues from the murine antibody sequence are grafted into the similar human “acceptor” germline. Subsequently, one or more positions near the CDRs or within the framework (e.g., Vernier positions) may be reverted to the original murine amino acid in order to achieve a humanized antibody with similar binding affinity to the original murine antibody. Typically, several versions of humanized antibodies with different reversion mutations are generated and empirically tested for activity. The humanized antibody variant with properties most similar to the parent murine antibody and the fewest murine framework reversions is selected as the final humanized antibody candidate.

### II. Composition

Applicant has discovered highly active antibodies that show high specificity for human coronaviruses (e.g., SARS-CoV-2). Accordingly, in various embodiments, the antibody or antigen-binding fragment thereof can selectively bind to a coronavirus. The antibodies and antigen-binding fragments described herein can have important applications, for both therapeutic and prophylactic treatment of coronavirus infections (e.g., COVID-19).

In summary, mAbs were synthesized that are clonally related and bind coronaviruses (e.g., SARS CoV-2). These antibodies are highly active neutralizers of coronavirus (e.g., SARS CoV-2) in vitro and provide broad protection from mortality and morbidity in vivo. The present disclosure provides knowledge about the binding mode and epitope of these mAbs which guides the development of universal COVID-19 vaccines.

The antibodies disclosed herein can be described or specified in terms of the epitope(s) that they recognize or bind. The portion of a target polypeptide that specifically interacts with the antigen binding domain of an antibody is an “epitope.” Furthermore, it should be noted that an “epitope” on can be a linear epitope or a conformational epitope, and in both instances can include non-polypeptide elements, e.g., an epitope can include a carbohydrate or lipid side chain. The term “affinity” refers to a measure of the strength of the binding of an individual epitope with an antibody's antigen binding site. In some embodiments, the epitope is an epitope in a coronavirus spike protein. In one aspect, the epitope is a conserved epitope proximal to the receptor binding motif (RBM). In a particular aspect, an epitope proximal to the RBM is an epitope centered on residues K444 and G446 of a coronavirus spike protein. In other aspect, an epitope is within amino acid residues 344-500 of a coronavirus spike protein.

An “anti-coronavirus spike antibody,” as used herein, refers to an isolated antibody that binds to recombinant human coronavirus spike protein or human coronavirus spike protein isolated from biological sample with an affinity constant or affinity of interaction (KD) between about 0.1 pM to about 10 μM, preferably about 0.1 pM to about 1 μM, more preferably about 0.1 pM to about 100 nM. Methods for determining the affinity of an antibody for an antigen are known in the art. Anti-coronavirus spike antibodies useful herein include those which are suitable for administration to a subject in a therapeutic amount.

Anti-coronavirus spike antibodies disclosed herein can also be described or specified in terms of their cross-reactivity. The term “cross-reactivity” refers to the ability of an antibody, specific for one antigen, to react with a second antigen; a measure of relatedness between two different antigenic substances. Thus, an antibody is cross-reactive if it binds to an epitope other than the one that induced its formation. The cross-reactive epitope generally contains many of the same complementary structural features as the inducing epitope, and in some cases, can actually fit better than the original. For example, certain antibodies have some degree of cross-reactivity, in that they bind related, but non-identical epitopes, e.g., epitopes with at least about 85%, at least about 90%, or at least about 95% identity (as calculated using methods known in the art) to a reference epitope. An antibody can be said to have little or no cross-reactivity if it does not bind epitopes with less than about 95%, less than about 90%, or less than about 85% identity to a reference epitope. An antibody can be deemed “highly specific” for a certain epitope, if it does not bind any other analog, ortholog, or homolog of that epitope.

In an exemplary embodiment, an anti-coronavirus spike antibody comprises a VL that has one or more HVRs derived from SEQ ID NO: 6 or a VH that has one or more HVRs derived from SEQ ID NO: 7. The HVR derived from SEQ ID NO: 6 may be L1, L2, L3, or any combination thereof. In certain embodiments, the VL may comprise an L1 of SEQ ID NO: 1, an L2 of DTS, an L3 of SEQ ID NO: 2, or any combination thereof (e.g. antibodies 1-7 in Table A). The HVR derived from SEQ ID NO: 7 may be H1, H2, H3, or any combination thereof. In certain embodiments, the VH may comprise an H1 of SEQ ID NO: 3, an H2 of SEQ ID NO: 4, an H3 of SEQ ID NO: 5, or any combination thereof (e.g. antibodies 8-14 in Table A). The antibody comprising one or more HVRs derived from SEQ ID NO: 7 may further comprise a light chain variable region (VL) comprising one or more HVRs derived from SEQ ID NO: 6. The HVR may be L1, L2, L3, or any combination thereof. In a preferred embodiment, the VL may comprise an L1 of SEQ ID NO: 1, an L2 of DTS, an L3 of SEQ ID NO: 2, or any combination thereof (e.g. antibodies 15-63 in Table A). In various embodiments above, the antibody may be a humanized antibody, or the antibody may have a VL with 90, 91, 92, 93, 94, 95, 96, 97, 98, 99, or 100% identity to SEQ ID NO: 6 and/or a VH with 90, 91, 92, 93, 94, 95, 96, 97, 98, 99 or 100% identity to SEQ ID NO: 7. In each of the above embodiments, the antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences of SEQ ID NO: 1, 2, 3, 4, 5, 6, 7, and the amino acid sequence DTS, which can readily be determined by one of skill in the art, and may be incorporated into a vector or other large DNA molecule, such as a chromosome, in order to express an antibody of the disclosure.

In various embodiments above, the antibody may have a conservative amino acid substitution to one or more residues of L1 of SEQ ID NO: 1, L2 of DTS, L3 of SEQ ID NO: 2, or any combination thereof and/or H1 of SEQ ID NO: 3, H2 of SEQ ID NO: 4, H3 of SEQ ID NO: 5 or any combination thereof. The in certain embodiments, the antibody of the disclosure may have a L1 with 90, 91, 92, 93, 94, 95, 96, 97, 98, 99, or 100% identity to SEQ ID NO: 1, a L2 with 90, 91, 92, 93, 94, 95, 96, 97, 98, 99, or 100% identity to DTS, a L3 with 90, 91, 92, 93, 94, 95, 96, 97, 98, 99, or 100% identity to SEQ ID NO: 2, a H1 with 90, 91, 92, 93, 94, 95, 96, 97, 98, 99, or 100% identity to SEQ ID NO: 3, a H2 with 90, 91, 92, 93, 94, 95, 96, 97, 98, 99, or 100% identity to SEQ ID NO: 4, and/or a H3 with 90, 91, 92, 93, 94, 95, 96, 97, 98, 99, or 100% identity to SEQ ID NO: 5.

TABLE A
Exemplary Antibodies

| | Light Chain HVR | | | Heavy Chain HVR | | |
|---|---|---|---|---|---|---|
| Antibody | L1 | L2 | L3 | H1 | H2 | H3 |
| 1 | SEQ ID NO: 1 | | | | | |
| 2 | SEQ ID NO: 1 | DTS | | | | |
| 3 | SEQ ID NO: 1 | DTS | SEQ ID NO: 2 | | | |
| 4 | | DTS | | | | |
| 5 | | DTS | SEQ ID NO: 2 | | | |
| 6 | | | SEQ ID NO: 2 | | | |
| 7 | SEQ ID NO: 1 | | SEQ ID NO: 2 | | | |
| 8 | | | | SEQ ID NO: 3 | | |
| 9 | | | | SEQ ID NO: 3 | SEQ ID NO: 4 | |
| 10 | | | | SEQ ID NO: 3 | SEQ ID NO: 4 | SEQ ID NO: 5 |
| 11 | | | | | SEQ ID NO: 4 | |
| 12 | | | | | SEQ ID NO: 4 | SEQ ID NO: 5 |
| 13 | | | | | | SEQ ID NO: 5 |
| 14 | | | | SEQ ID NO: 3 | | SEQ ID NO: 5 |
| 15 | SEQ ID NO: 1 | | | SEQ ID NO: 3 | | |
| 16 | SEQ ID NO: 1 | | | SEQ ID NO: 3 | SEQ ID NO: 4 | |
| 17 | SEQ ID NO: 1 | | | SEQ ID NO: 3 | SEQ ID NO: 4 | SEQ ID NO: 5 |
| 18 | SEQ ID NO: 1 | | | | SEQ ID NO: 4 | |
| 19 | SEQ ID NO: 1 | | | | SEQ ID NO: 4 | SEQ ID NO: 5 |
| 20 | SEQ ID NO: 1 | | | | | SEQ ID NO: 5 |
| 21 | SEQ ID NO: 1 | | | SEQ ID NO: 3 | | SEQ ID NO: 5 |
| 22 | SEQ ID NO: 1 | DTS | | SEQ ID NO: 3 | | |
| 23 | SEQ ID NO: 1 | DTS | | SEQ ID NO: 3 | SEQ ID NO: 4 | |
| 24 | SEQ ID NO: 1 | DTS | | SEQ ID NO: 3 | SEQ ID NO: 4 | SEQ ID NO: 5 |
| 25 | SEQ ID NO: 1 | DTS | | | SEQ ID NO: 4 | |
| 26 | SEQ ID NO: 1 | DTS | | | SEQ ID NO: 4 | SEQ ID NO: 5 |
| 27 | SEQ ID NO: 1 | DTS | | | | SEQ ID NO: 5 |
| 28 | SEQ ID NO: 1 | DTS | | SEQ ID NO: 3 | | SEQ ID NO: 5 |
| 29 | SEQ ID NO: 1 | DTS | SEQ ID NO: 2 | SEQ ID NO: 3 | | |
| 30 | SEQ ID NO: 1 | DTS | SEQ ID NO: 2 | SEQ ID NO: 3 | SEQ ID NO: 4 | |
| 31 | SEQ ID NO: 1 | DTS | SEQ ID NO: 2 | SEQ ID NO: 3 | SEQ ID NO: 4 | SEQ ID NO: 5 |
| 32 | SEQ ID NO: 1 | DTS | SEQ ID NO: 2 | | SEQ ID NO: 4 | |
| 33 | SEQ ID NO: 1 | DTS | SEQ ID NO: 2 | | SEQ ID NO: 4 | SEQ ID NO: 5 |
| 34 | SEQ ID NO: 1 | DTS | SEQ ID NO: 2 | SEQ ID NO: 3 | | SEQ ID NO: 5 |
| 35 | SEQ ID NO: 1 | DTS | SEQ ID NO: 2 | | | SEQ ID NO: 5 |
| 36 | | DTS | | SEQ ID NO: 3 | | |
| 37 | | DTS | | SEQ ID NO: 3 | SEQ ID NO: 4 | |
| 38 | | DTS | | SEQ ID NO: 3 | SEQ ID NO: 4 | SEQ ID NO: 5 |
| 39 | | DTS | | | SEQ ID NO: 4 | |
| 40 | | DTS | | | SEQ ID NO: 4 | SEQ ID NO: 5 |
| 41 | | DTS | | | | SEQ ID NO: 5 |
| 42 | | DTS | | SEQ ID NO: 3 | | SEQ ID NO: 5 |
| 43 | | DTS | SEQ ID NO: 2 | SEQ ID NO: 3 | | |
| 44 | | DTS | SEQ ID NO: 2 | SEQ ID NO: 3 | SEQ ID NO: 4 | |
| 45 | | DTS | SEQ ID NO: 2 | SEQ ID NO: 3 | SEQ ID NO: 4 | SEQ ID NO: 5 |
| 46 | | DTS | SEQ ID NO: 2 | | SEQ ID NO: 4 | |
| 47 | | DTS | SEQ ID NO: 2 | | SEQ ID NO: 4 | SEQ ID NO: 5 |
| 48 | | DTS | SEQ ID NO: 2 | | | SEQ ID NO: 5 |
| 49 | | DTS | SEQ ID NO: 2 | SEQ ID NO: 3 | | SEQ ID NO: 5 |
| 50 | | | SEQ ID NO: 2 | SEQ ID NO: 3 | | |
| 51 | | | SEQ ID NO: 2 | SEQ ID NO: 3 | SEQ ID NO: 4 | |
| 52 | | | SEQ ID NO: 2 | SEQ ID NO: 3 | SEQ ID NO: 4 | SEQ ID NO: 5 |
| 53 | | | SEQ ID NO: 2 | | SEQ ID NO: 4 | |
| 54 | | | SEQ ID NO: 2 | | SEQ ID NO: 4 | SEQ ID NO: 5 |
| 55 | | | SEQ ID NO: 2 | | | SEQ ID NO: 5 |
| 56 | | | SEQ ID NO: 2 | SEQ ID NO: 3 | | SEQ ID NO: 5 |
| 57 | SEQ ID NO: 1 | | SEQ ID NO: 2 | SEQ ID NO: 3 | | |
| 58 | SEQ ID NO: 1 | | SEQ ID NO: 2 | SEQ ID NO: 3 | SEQ ID NO: 4 | |
| 59 | SEQ ID NO: 1 | | SEQ ID NO: 2 | SEQ ID NO: 3 | SEQ ID NO: 4 | SEQ ID NO: 5 |
| 60 | SEQ ID NO: 1 | | SEQ ID NO: 2 | | SEQ ID NO: 4 | |
| 61 | SEQ ID NO: 1 | | SEQ ID NO: 2 | | SEQ ID NO: 4 | SEQ ID NO: 5 |
| 62 | SEQ ID NO: 1 | | SEQ ID NO: 2 | | | SEQ ID NO: 5 |
| 63 | SEQ ID NO: 1 | | SEQ ID NO: 2 | SEQ ID NO: 3 | | SEQ ID NO: 5 |

TABLE B
Illustrative Sequences for Anti-SARS-CoV-2
antibodies

| | | | SEQ ID |
|---|---|---|---|
| Antibody | Description | Amino Acids | NO: |
| SARS2-38 | L1 | STVSF | 1 |
| SARS2-38 | L2 | DTS | |
| SARS2-38 | L3 | QQWNTYPLT | 2 |
| SARS2-38 | H1 | GFSLTRYG | 3 |
| SARS2-38 | H2 | IWADGST | 4 |
| SARS2-38 | H3 | ARDGRGYDDY | 5 |
| SARS2-38 | VL | QIVLTQSPAIMSASPGEKVTMT | 6 |
| | | CSASSTVSFIYWYQQKPGSSP | |
| | | RLLIYDTSNPASGVPVRFSGSG | |
| | | CGTSYYLTISRMEAEDAATYYC | |
| | | QQWNTYPLTFGAGTKLELK | |
| SARS2-38 | VH | QVQLKESGPGLVAPSQSLSITC | 7 |
| | | TVSGFSLTRYGVHWVRQPPGK | |
| | | GLEWLGVIWADGSTYYNSALM | |
| | | SRLSISKDNSKSQVFLNMNSLQ | |
| | | TDDTAKYYCARDGRGYDDYW | |
| | | GQGTTLTVSS | |

Also provided are peptides, polypeptides and/or proteins derived from any of the antibodies or antibody binding fragments described herein. Generally, as used herein, the derivatives provided here are substantially similar to the antibodies or antibody binding fragments described herein. For example, they may contain one or more conservative substitutions in their amino acid sequences or may contain a chemical modification. The derivatives and modified peptides/polypeptides/proteins all are considered “structurally similar” which means they retain the structure (e.g., the secondary, tertiary or quarternary structure) of the parent molecule and are expected to interact with the antigen in the same way as the parent molecule.

A class of synthetically derived antibodies or antigen-binding moieties can be generated by conservatively mutating resides on the parent molecule to generate a peptide, polypeptide or protein maintaining the same activity as the parent molecule. Representative conservative substitutions are known in the art and are also summarized here.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

A second way to generate a functional peptide/polypeptide or protein based on the sequences provided herein is through the use of computational, “in-silico” design. For example, computationally designed antibodies or antigen-binding fragments may be designed using standard methods of the art. For example, see Strauch E M et al., (Nat Biotechnol. 2017 July;35(7):667-671), Fleishman S J et al., (Science. 2011 May 13;332(6031):816-21), and Koday M T et al., (PLoS Pathog. 2016 Feb. 4;12(2):e1005409), each incorporated by reference in their entirety.

In various embodiments, an antibody or antibody binding fragment thereof is provided that binds a coronavirus (e.g., SARS-CoV-2) and is structurally similar to any of the antibodies described herein. That is it has the same secondary, tertiary or quaternary structure as the antibodies or antigen-binding fragments described herein. For example, the antibody or antigen-binding fragment can have a tertiary structure that is structurally similar to a single CDR loop. For example, the antibody or antigen-binding fragment can have a tertiary structure that is structurally similar to a H3 loop, e.g., a loop comprising those disclosed in Table A and/or Table B or any combination thereof. Alternatively or in addition, the antibody or antigen-binding fragment can have a tertiary structure that is structurally similar to a CDR loop comprising any one of those disclosed in Table A and/or Table B. In one embodiment, an antibody of the disclosure competitively inhibits SARS2-38 binding to recombinant s protein (e.g. binding to a S protein comprising amino acid SEQ ID NO: 8).

A test antibody is said to competitively inhibit binding of a reference antibody (e.g., those discussed above, SARS2-38) to a given epitope if the test antibody preferentially binds to that epitope to the extent that it blocks binding of the reference antibody to the epitope by at least 50%, at least 60%, at least 70%, at least 80%, or at least 90%. Competitive inhibition can be determined by any method known in the art. In a specific example, competitive inhibition is determined by a competitive inhibition ELISA comprising the steps of: coating a binding surface or support with a purified reference epitope-binding agent to form an antibody coated surface; combining a predetermined amount of a purified, labeled antigen and a test sample containing a test antibody; adding the incubated mixture of labeled antigen and test antibody to said coated surface; incubating said coated surface with said combination of antigen and test antibody; and measuring the amount of antigen-binding inhibition as compared to conditions that lack the test antibody.

In various embodiments, the antibody can comprise at least one amino acid substitution, deletion, or insertion in a variable region, a hinge region or an Fc region relative to the sequence of a wild-type variable region, hinge region or a wild-type Fc region.

For example, the antibody can comprise an Fc region that contains at least one amino acid substitution, deletion, or insertion relative to the sequence of a wild-type Fc region. In various embodiments, this substitution, deletion or insertion can prevent or reduce recycling of the antibody (e.g., in vivo).

In various embodiments, the antibody or antigen-binding fragment can comprise a heavy chain variable region and/or light chain variable region comprising at least one amino acid substitution, deletion, or insertion as compared to any one of the antibodies disclosed in Table A or Table B.

Further, as described further below, the antibodies or antigen-binding fragments described herein can be expressed recombinantly (e.g., using a recombinant cell line or recombinant organism). Accordingly, the antibodies or antigen-binding fragments may comprise post-translational modifications (e.g., glycosylation profiles, methylation) that differs from naturally occurring antibodies.

The antibodies and antigen-binding fragments thereof described herein have some measure of binding affinity to a coronavirus. Most preferably, the antibody or antigen-binding fragment binds SARS-CoV-2 (that is, the coronavirus comprises SARS-CoV-2). In various embodiments, the antibodies and antigen-binding fragments thereof described herein can bind an epitope proximal to the receptor binding domain (RBD) expressed by the coronavirus (e.g., SARS-CoV-2). In one aspect, the epitope is a conserved epitope proximal to the receptor binding domain (RBD). In a particular aspect, an epitope proximal to the RBM is an epitope centered on residues K444 and G446 of a coronavirus spike protein. In another specific embodiment, an epitope is within amino acid residues 344-500 of a coronavirus spike protein (e.g. SEQ ID NO: 8).

The binding of the antibody or antigen-binding fragment can neutralize the coronavirus (e.g., SARS-CoV-2). In various embodiments, the antibodies and/or binding fragment neutralize the coronavirus with an IC₅₀ of about 0.0001 μg/ml to about 30 μg/ml. For example, the antibody or antigen-binding fragment can have an IC₅₀ of about 0.001 μg/ml to about 30 μg/ml. The neutralizing ability of the antibody or antigen-binding fragment can be determined by measuring, for example, the ability of the virus to replicate in the presence or absence of the antibody or antigen-binding fragment.

In various embodiments, the antibody or antigen-binding fragment described herein is humanized. “Humanized” antibodies are generally chimeric or mutant monoclonal antibodies from mouse, rat, hamster, rabbit or other species, bearing human constant and/or variable region domains or specific changes.

In various embodiments, the antibody or antigen-binding fragment described herein is a monoclonal antibody. As used herein, the term “monoclonal antibodies” refer to antibodies or antigen-binding fragments that are expressed from the same genetic sequence or sequences and consist of identical antibody molecules.

In various embodiments, the antibody or antigen-binding fragment described herein is an IgG type antibody. For example, the antibody or antigen-binding fragment can be an IgG1, IgG2, IgG3, or an IgG4 type antibody.

DNA molecules encoding light chain variable regions and/or heavy chain variable regions can be chemically synthesized. Synthetic DNA molecules can be ligated to other appropriate nucleotide sequences, including, e.g., constant region coding sequences, and expression control sequences, to produce conventional gene expression constructs encoding the desired antibody. Production of defined gene constructs is within routine skill in the art.

Nucleic acids encoding desired antibodies can be incorporated (ligated) into expression vectors, which can be introduced into host cells through conventional transfection or transformation techniques. Illustrative host cells are E. coli cells, Chinese hamster ovary (CHO) cells, HeLa cells, baby hamster kidney (BHK) cells, monkey kidney cells (COS), human hepatocellular carcinoma cells (e.g., Hep G2), human embryonal kidney (HEK) cells and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. This expression vector optionally contains sequences encoding all or part of a constant region, enabling an entire, or a part of, a heavy or light chain to be expressed. The gene construct can be introduced into eukaryotic host cells using conventional techniques. The host cells express VL or VH fragments, VL-VH heterodimers, VH-VL or VL-VH single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector encoding a polypeptide comprising an entire, or part of, a heavy chain or heavy chain variable region, and another expression vector encoding a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such variable region, under conditions that permit expression of the polypeptide. Following expression, the polypeptide can be harvested and purified or isolated using techniques, e.g., using affinity tags such as glutathione-S-transferase (GST) and histidine tags.

A monoclonal antibody, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit ex-pression of both chains. The intact antibody (or antigen-binding fragment of the antibody) can be harvested and purified or isolated using other techniques, e.g., Protein A, Protein G, affinity tags such as glutathione-S-transferase (GST) and histidine tags. The heavy chain and the light chain can be expressed from a single expression vector or from two separate expression vectors.

Therefore, in various embodiments, a nucleic acid is provided, the nucleic acid comprising a nucleotide sequence encoding the antibody or antigen-binding fragment described herein. The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules.

Suitable nucleic acids that can encode portions of the inventive antibodies can be determined using standard techniques. In various embodiments, the nucleic acid comprises a nucleotide sequence encoding an immunoglobulin heavy chain variable region of the antibody or antigen-binding fragment described herein. In various embodiments, the nucleic acid comprises a nucleotide sequence encoding an immunoglobulin light chain variable region of the antibody or antigen-binding fragment described herein. In some embodiments, the nucleic acids encode one or more complementary determining regions (CDR) having the amino acid sequences described herein. As described above, a single nucleic acid may be provided that encodes more than one protein product (e.g., the immunoglobulin light chain and the immunoglobulin heavy chain). Alternatively, two or more separate nucleic acids may be provided each encoding one component of the antibody and/or antigen-binding fragment (e.g., the light chain or the heavy chain).

In various embodiments, an expression vector is provided comprising one or more of the nucleic acids described herein. Vectors can be derived from plasmids such as: F, F1, RP1, Col, pBR322, TOL, Ti, etc; cosmids; phages such as lambda, lambdoid, M13, Mu, P1, P22, Qβ, T-even, T-odd, T2, T4, T7 etc; or plant viruses. Vectors can be used for cloning and/or expression of the binding molecules of the invention and might even be used for gene therapy purposes. Vectors comprising one or more nucleic acid molecules according to the invention operably linked to one or more expression-regulating nucleic acid molecules are also covered by the present invention. The choice of the vector is dependent on the recombinant procedures followed and the host used. Introduction of vectors in host cells can be affected by inter alia calcium phosphate transfection, virus infection, DEAE-dextran mediated transfection, lipofectamine transfection or electroporation. Vectors may be autonomously replicating or may replicate together with the chromosome into which they have been integrated. Preferably, the vectors contain one or more selection markers. The choice of the markers may depend on the host cells of choice. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the human binding molecules as described above operably linked to one or more nucleic acid molecules encoding proteins or peptides that can be used to isolate the human binding molecules are also covered by the invention. These proteins or peptides include, but are not limited to, glutathione-S-transferase, maltose binding protein, metal-binding polyhistidine, green fluorescent protein, luciferase and beta-galactosidase.

The expression vector may be transfected into a host cell to induce the translation and expression of the nucleic acid into the heavy chain variable region and/or the light chain variable region. Therefore, a host cell is provided comprising any expression vector described herein. Host cells include, but are not limited to, cells of mammalian, plant, insect, fungal or bacterial origin. Bacterial cells include, but are not limited to, cells from Gram-positive bacteria or Gram-negative bacteria such as several species of the genera Escherichia, such as E. coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as inter alia cells from crop plants such as forestry plants, or cells from plants providing food and raw materials such as cereal plants, or medicinal plants, or cells from ornamentals, or cells from flower bulb crops. Transformed (transgenic) plants or plant cells are produced by methods such as Agrobacterium-mediated gene trans-fer, transformation of leaf discs, protoplast transformation by polyethylene glycol-induced DNA transfer, electroporation, sonication, microinjection or bolistic gene transfer. Additionally, a suitable expression system can be a baculovirus system. Expression systems using mammalian cells, such as Chinese Hamster Ovary (CHO) cells, COS cells, BHK cells, NSO cells or Bowes melanoma cells are preferred in the present invention. Since the present invention deals with molecules that may have to be administered to humans, a completely human expression system would be particularly preferred. Therefore, even more preferably, the host cells are human cells. Examples of human cells are, inter alia, HeLa, 911, AT1080, A549, HEK293, 293F and HEK293T cells.

Accordingly, the antibody or antigen-binding fragment can be expressed using a recombinant cell line or recombinant organism.

Further a method is provided for producing an antibody or antigen-binding fragment that binds a coronavirus, the method comprising growing a host cell as described herein under conditions so that the host cell expresses a polypeptide or polypeptides comprising the immunoglobulin heavy chain variable region and the immunoglobulin light chain variable region, thereby producing the antibody or antigen-binding fragment and purifying the antibody or antigen-binding fragment.

Also provided are pharmaceutical compositions comprising at least one antibody or antigen-binding fragment described herein.

Pharmaceutical compositions containing one or more of the antibodies or antigen-binding fragments described herein can be formulated in any conventional manner. Proper formulation is dependent in part upon the route of administration selected. Routes of administration include, but are not limited to parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. Preferably, the composition is administered parenterally or is inhaled (e.g., intranasal). For example, the composition can be administered by intravenous infusion.

The pharmaceutical compositions can be formulated for parenteral administration, e.g., formulated for injection via intravenous, intra-arterial, subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

The pharmaceutical composition can be formulated without blood, plasma or a major component of blood or plasma (e.g., blood cells, fibrin, hemoglobin, albumin, etc.).

The pharmaceutical composition can comprise from about 0.001 to about 99.99 wt. % of the antibody or antigen-binding fragment according to the total weight of the composition. For example, the pharmaceutical composition can comprise from about 0.001 to about 1%, about 0.001 to about 5%, about 0.001 to about 10%, about 0.001 to about 15%, about 0.001 to about 20%, about 0.001 to about 25%, about 0.001 to about 30%, about 1 to about 10%, about 1 to about 20%, about 1 to about 30%, about 10 to about 20%, about 10 to about 30%, about 10 to about 40%, about 10 to about 50%, about 20 to about 30%, about 20 to about 40%, about 20 to about 50%, about 20 to about 60%, about 20 to about 70%, about 20 to about 80%, about 20 to about 90%, about 30 to about 40%, about 30 to about 50%, about 30 to about 60%, about 30 to about 70%, about 30 to about 80%, about 30 to about 90%, about 40 to about 50%, about 40 to about 60%, about 40 to about 70%, about 40 to about 80%, about 40 to about 90%, about 50 to about 99.99%, about 50 to about 99%, about 60 to about 99%, about 70 to about 99%, about 80 to about 99%, about 90 to about 99%, about 50 to about 95%, about 60 to about 95%, about 70 to about 95%, about 80 to about 95%, about 90 to about 95%, about 50 to about 90%, about 60 to about 90%, about 70 to about 90%, about 80 to about 90%, about 85 to about 90%, about 50 to about 80%, about 60 to about 80%, about 70 to about 80%, about 75 to about 80%, about 50 to about 70%, about 60 to about 70%, or from about 50 to about 60% of the antibody or antigen-binding fragment by weight according to the total weight of the composition.

The compositions described herein can also comprise one or more pharmaceutically acceptable excipients and/or carriers. The pharmaceutically acceptable excipients and/or carriers for use in the compositions of the present invention can be selected based upon a number of factors including the particular compound used, and its concentration, stability and intended bioavailability; the subject, its age, size and general condition; and the route of administration.

Some examples of materials which can serve as pharmaceutically acceptable carriers in the compositions described herein are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, nonvolatile silicones, adhesives, bulking agents, flavorings, sweeteners, adsorbents, binders, disintegrating agents, lubricants, coating agents, and antioxidants.

In some embodiments, the composition further comprises at least one other therapeutic, prophylactic and/or diagnostic agent. Preferably, the therapeutic and/or prophylactic agents are capable of preventing and/or treating a coronavirus infection and/or a condition/symptom resulting from such an infection. Therapeutic and/or prophylactic agents include, but are not limited to, antiviral agents. Such agents can be binding molecules, small molecules, organic or inorganic compounds, enzymes, polynucleotide sequences, antiviral peptides, etc. The therapeutic and/or prophylactic agent can comprise an M2 inhibitor (e.g., amantadine, rimantadine) and/or a neuraminidase inhibitor (e.g., zanamivir, oseltamivir). In various embodiments, the anti-viral agent can comprise baloxavir, oseltamivir, zanamivir, peramivir, remdesivir, or any combination thereof. The therapeutic and/or prophylactic agent can also include various anti-malarial such as chloroquine, hydroxychloroquine, and analogues thereof.

The additional antibodies or therapeutic/prophylactic and/or diagnostic agents may be used in combination with the antibodies and antigen-binding fragments of the present invention. “In combination” herein, means simultaneously, as separate formulations (e.g., co-administered), or as one single combined formulation or according to a sequential administration regiment as separate formulations, in any order. Agents capable of preventing and/or treating an infection with coronavirus (e.g., SARS-CoV-2) and/or a condition resulting from such an infection that are in the experimental phase might also be used as other therapeutic and/or prophylactic agents useful in the present invention.

In various embodiments a vaccine is provided for preventing a coronavirus infection. Advantageously the vaccine can provide protection from SARS CoV-2 which can cause an infection known as COVID-19. In various embodiments, the vaccine may comprise a polypeptide comprising the epitope targeted by the antibodies or antigen-binding fragments described herein.

In various embodiments, the vaccine further comprises an adjuvant to stimulate an immune response. Suitable adjuvants can include, for example, alum, aluminum hydroxide, monophosphoryl lipid A (MPL) or combinations thereof. Further, the vaccine may be prepared using suitable carriers and excipients according to pharmaceutical compositions described herein above.

In various embodiments, the vaccine can elicit an immunological response to prevent a coronavirus infection. The infection may be caused by the SARS-CoV-2 virus. For example, the infection can comprise COVID-19.

### II. Treatment Methods

The present disclosure encompasses methods to treat, prevent, or reduce the infectivity of a virus in a subject in need thereof. In some embodiments, the methods prevent or reduce the infectivity of a viral infection by preventing internalization of a virus into a cell of the subject or by preventing internalization of a viral genome into a cell of the subject. In some embodiments, administration of a composition provided herein, for instance those described in Section I, may disrupt or prevent an interaction between a viral surface protein (e.g., a spike protein) and a host receptor protein (e.g., an epithelial angiotensin converting enzyme (ACE)). For example, administration of a composition of the disclosure may block internalization of a coronavirus into a cell of a subject by blocking or disrupting interactions between a coronavirus spike protein and a host receptor protein and/or by sequestering the virus in vivo allowing for the virus bound to the composition to be eliminated by the subject's immune cells. Administering a composition of the disclosure to a subject at risk for a viral infection may reduce the risk of coronavirus infection in the subject.

In other embodiments, the present disclosure provides methods to treat, prevent, or reduce the infectivity of a respiratory viral infection. In some embodiments, the viral infection may be a coronavirus infection. The coronavirus may be SARS-CoV, SARS-CoV-2, MERS-CoV, HKU1, OC43, or 229E. The coronavirus may be a beta-coronavirus. A subject at risk for a coronavirus infection may come in contact with an asymptomatic carrier of the coronavirus infection, thereby unknowingly contracting the coronavirus infection.

In some embodiments, the compositions, methods, or treatment regiments disclosed herein may treat or prevent a SARS-CoV-2 infection (e.g., COVID-19). A SARS-CoV-2 infection may depend on host cell ACE-2 enzyme. In some embodiments, a SARS-CoV-2 infection may be blocked (e.g., prevented, treated, or slowed) by a composition of the disclosure. In various embodiments, a method of preventing or treating a coronavirus infection (e.g., COVID-19 caused by SARS-CoV-2) in a subject in need thereof is provided. The method can comprise administering any antibody or antigen-binding fragment (including any nucleic acid or expression vector that encodes the antibody or antigen-binding fragment), any vaccine, or any composition as described herein to the subject.

In various embodiments, the composition is administered parentally (e.g., systemically). In other embodiments, the composition is inhaled orally (e.g., intranasally). In both cases the composition is formulated (e.g., with carriers/excipients) according to its mode of administration as described above.

In various embodiments the composition is administered via intranasal, intramuscular, intravenous, and/or intradermal routes. In some embodiments, the composition is provided as an aerosol (e.g., for nasal administration).

Dosing regiments can be adjusted to provide the optimum desired response (e.g., a prophylactic or therapeutic response). Therefore, the dose used in the methods herein can vary depended on the intended use (e.g., for prophylactic vs. therapeutic use). Nevertheless, the compositions described herein may be administered at a dose of about 1 to about 100 mg/kg body weight, or from about 1 to about 70 mg/kg body weight. Furthermore, a single bolus may be administered, several divided doses may be administered over time, or the dose may be proportionally reduced or increased as indicated by the exigencies of the therapeutic of the therapeutic situation.

In various embodiments, the antibody, antigen-binding fragment, or S protein epitope is delivered using a gene therapy technique. Such techniques generally comprise administering a viral vector comprising a nucleic acid that codes for a gene product of interest to a subject in need thereof. Therefore, in certain embodiments, the antibody or antigen-binding fragment described herein is delivered to a subject in need thereof by administering a viral vector or vectors (e.g., an adenovirus) containing one or more of the necessary nucleic acids (such as, for example, the nucleic acids provided herein) for expressing the antibody or antibody binding fragment in vivo. Similar delivery methods have successfully lead to the expression of protective antibodies in other disease contexts. For example, see Sofer-Podesta C. et al., “Adenovirus-mediated delivery of an Anti-V Antigen Monoclonal Antibody Protects Mice against a Lethal Yersinia pestis Challenge” Infection and Immunity March 2009, 77 (4) 1561-1568, the entire disclosure of which is incorporated herein by reference.

In various embodiments, the coronavirus infection to be treated is a SARS infection (e.g., severe acute respiratory syndrome caused by the coronavirus). In various embodiments, the coronavirus infection comprises COVID-19.

Generally, the methods as described herein comprise administration of a therapeutically effective amount of a composition of the disclosure to a subject. The methods described herein are generally performed on a subject in need thereof. A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion ani-mal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

The concentration of antibody in formulations to be administered is an effective amount and ranges from as low as about 0.1% by weight to as much as about 15 or about 20% by weight and will be selected primarily based on fluid volumes, viscosities, and so forth, in accordance with the particular mode of administration selected if desired. A typical composition for injection to a living subject could be made up to contain 1 mL sterile buffered water of phosphate buffered saline and about 1-1000 mg of any one of or a combination of the antibodies disclosed herein. The formulation could be sterile filtered after making the formulation, or otherwise made microbiologically acceptable. A typical composition for intravenous infusion could have volumes between 1-250 mL of fluid, such as sterile Ringer's solution, and 1-100 mg per ml, or more in antibody of the disclosure concentration. Antibodies disclosed herein can be frozen or lyophilized for storage and reconstituted in a suitable sterile carrier prior to use. Lyophilization and reconstitution may lead to varying degrees of antibody activity loss (e.g. with conventional immune globulins, IgM antibodies tend to have greater activity loss than IgG antibodies). Dosages administered are effective dosages and may have to be adjusted to compensate. The pH of the formulations generally pharmaceutical grade quality, will be selected to balance antibody stability (chemical and physical) and comfort to the subject when administered. Generally, a pH between 4 and 8 is tolerated. Doses will vary from individual to individual based on size, weight, and other physiobiological characteristics of the individual receiving the successful administration.

As used herein, the term “therapeutically effective amount” means an amount of a substance (e.g. an antibody of the disclosure) that leads to measurable and beneficial effects for the subject administered the substance, i.e., significant efficacy. The therapeutically effective amount or dose of compound administered according to this discovery will be determined using standard clinical techniques and may be by influenced by the circumstances surrounding the case, including the antibody administered, the route of administration, and the status of the symptoms being treated, among other considerations. A typical dose may contain from about 0.01 mg/kg to about 100 mg/kg of an antibody of the disclosure described herein. Doses can range from about 0.05 mg/kg to about 50 mg/kg, more preferably from about 0.1 mg/kg to about 25 mg/kg. The frequency of dosing may be daily or once, twice, three times or more per week or per month, as needed as to effectively treat the symptoms.

The timing of administration of the treatment relative to the disease itself and duration of treatment will be determined by the circumstances surrounding the case. Duration of treatment could range from a single dose administered on a one-time basis to a life-long course of therapeutic treatments.

Although the foregoing methods appear the most convenient and most appropriate and effective for administration of proteins such as humanized antibodies, by suitable adaptation, other effective techniques for administration, such as intraventricular administration, transdermal administration and oral administration may be employed provided proper formulation is utilized herein. In addition, a person skilled in the art can use a polynucleotide of the invention encoding any one of the above-described antibodies instead of the proteinaceous material itself. For example,

In addition, it may be desirable to employ controlled release formulations using biodegradable films and matrices, or osmotic mini-pumps, or delivery systems based on dextran beads, alginate, or collagen.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—A Potently Neutralizing Anti-SARS-CoV-2 Antibody Inhibits Variants of Concern by Binding a Highly Conserved Epitope

Severe acute respiratory syndrome-related coronavirus (SARS-CoV) and SARS-CoV-2 belong to the Sarbecovirus subgenus of Betacoronaviruses. In little more than a year, the coronavirus disease 2019 (COVID-19) pandemic caused by the rapid emergence of SARS-CoV-2 has resulted in over 140 million infections and 3 million deaths worldwide. Multiple effective vaccines against SARS-CoV-2 that prevent COVID-19 have been rapidly developed and deployed. Monoclonal antibodies (mAb) also have shown efficacy in animal models of SARS-CoV-2 infection, and two mAb treatments are approved for use in patients under Emergency Use Authorization (EUA). Therapy with mAbs may be beneficial to high-risk patients following exposure to SARS-CoV-2 with mild or moderate symptoms, but prior to onset of severe disease signs and symptoms, and can complement the usage of vaccines as a means of combating the COVID-19 pandemic.

The majority of characterized potently neutralizing and protective anti-SARS-CoV-2 mAbs bind the receptor binding domain (RBD) of the viral spike protein, though some inhibitory mAbs against the N-terminal domain (NTD) of spike also have been described. Under immune selection pressure, SARS-CoV-2 can select for mutations in the RBD and NTD that enable escape from antibody recognition and neutralization. Indeed, several emerging SARS-CoV-2 variants have mutations in the spike protein, including the RBD and NTD, that confer resistance to mAbs or polyclonal antibodies (pAbs) elicited by vaccines or natural infection. As such, additional mAbs or vaccines that retain efficacy against emerging SARS-CoV-2 variants may be needed to combat new and evolving strains.

In the present Example, a panel of potently neutralizing murine mAbs against the RBD of SARS-CoV-2 is described that bind several epitopes proximal to the receptor binding motif (RBM) of the RBD or at the base of the RBD. Although some neutralizing mAbs demonstrated limited ability to protect against infection by the historical SARS-CoV-2 WA1/2020 strain in a mouse disease model and selected for rapid escape in vivo, others protected completely in the context of prophylactic or therapeutic administration. Two protective mAbs, SARS2-02 and SARS2-38, showed variable capacity to neutralize variants of concern (VOCs): SARS2-02 binds an epitope that includes residues E484 and L452 and has reduced potency against strains (B.1.429, B.1.351, and B.1.1.28) encoding these mutations. In contrast, SARS2-38 binds an epitope centered on residues K444 and G446 and potently neutralized all tested VOCs. Analysis of a cryo-electron microscopy (cryo-EM) structure of SARS2-38 bound to spike reveals that this mAb binds a conserved epitope on the RBD that is also engaged, albeit through distinct geometries, by other neutralizing and protective human mAbs. Thus, treatment with mAbs or induction of pAbs targeting this conserved region of the RBD confer protection against many emerging SARS-CoV-2 variants.

### RESULTS

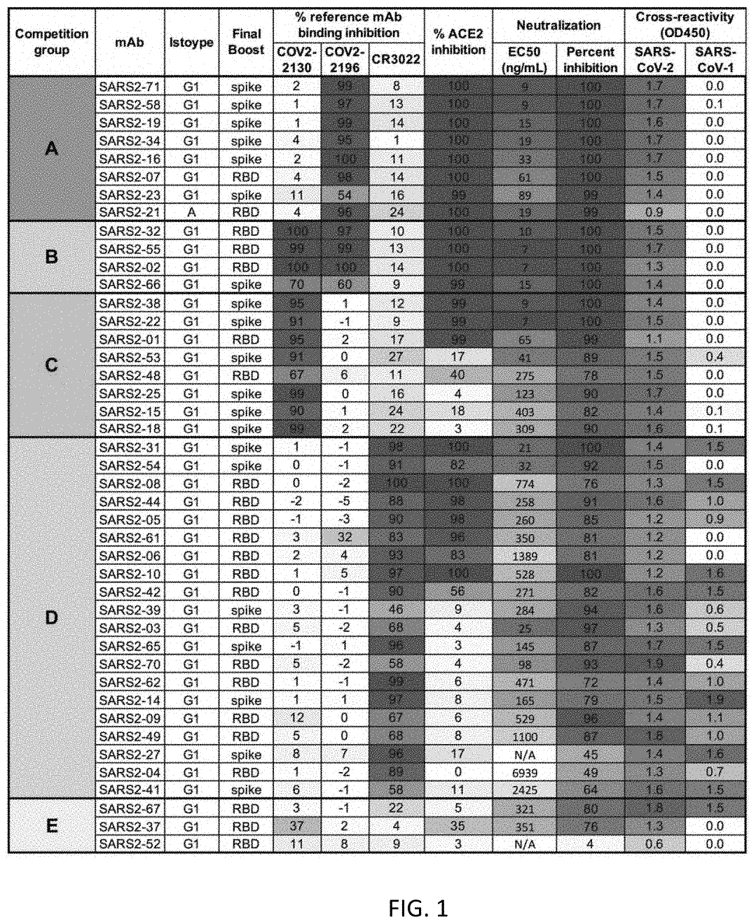

Development and characterization of anti-SARS-CoV-2 mAbs: A panel of anti-SARS-CoV-2 mAbs was generated from BALB/c mice that were immunized and boosted with purified RBD and/or ectodomain of the spike protein mixed with AddaVax™, a squalene-based adjuvant (FIG. 1). After splenocyte-myeloma fusions, hybridoma supernatants were screened for antibody binding to recombinant spike protein and permeabilized SARS-CoV-2-infected Vero cells by ELISA and flow cytometry, respectively. Sixty-four hybridomas producing anti-SARS-CoV-2 antibodies were cloned by limiting dilution. Forty-three of these mAbs bound to recombinant RBD and were selected for further study because prior experiments showed this class included potently inhibitory antibodies; the majority of these mAbs were of the IgG1 subclass (FIG. 1).

The mAbs were evaluated by competition binding analysis using three previously characterized human mAbs that recognize distinct antigenic sites on the RBD (COV2-2196, COV2-2130, and CR3022) (FIG. 1). Eight mAbs competed for spike protein binding with the neutralizing mAb COV2-2196 only, eight mAbs competed with the neutralizing mAb COV2-2130 only, four mAbs competed with both COV2-2196 and COV2-2130, and twenty mAbs competed with CR3022, which recognizes a more conserved, cryptic, and non-neutralizing epitope on the SARS-CoV-2 spike protein distal from the receptor binding site. Three RBD-binding mAbs did not compete with COV2-2196, COV2-2130, or CR3022. Based on the binding analysis, mAbs were divided into 5 competition groups, A-E (FIG. 1).

One potential mechanism of antibody-mediated neutralization of SARS-CoV-2 is through inhibition of viral spike protein binding to the human ACE2 receptor. The COV2-2196 epitope directly overlaps the ACE2 binding site on RBD, whereas the COV2-2130 epitope lies proximal to residues in the RBM that interact with ACE2; nonetheless, both mAbs can block spike binding to ACE2. In contrast, CR3022 engages the base of the RBD and does not block ACE2 binding to spike. Of the 43 RBD-binding antibodies in our panel, all mAbs in groups A and B inhibited ACE2 binding to spike protein, mAbs in groups C and D variably inhibited ACE2 binding, and mAbs in group E failed to inhibit ACE2 binding (FIG. 1).

The mAbs also were tested for cross-reactive binding to the SARS-CoV-1 spike protein. The majority of mAbs in group D, which competed with the cross-reactive mAb CR3022 for spike binding, cross-reacted with SARS-CoV-1 spike protein, indicating they bind conserved sarbecovirus epitopes. MAbs in groups A, B, and C did not bind to SARS-CoV-1 (FIG. 1), and one group E mAb recognized SARS-CoV-1.

Neutralizing activity of anti-SARS-CoV-2 mAbs: Next, the neutralizing activity of mAb hybridoma supernatants were determined using a focus-reduction neutralization test (FRNT) and Vero E6 cells with the WA1/2020 SARS-CoV-2 strain. Antibody concentrations in the supernatants were quantified by ELISA and used to calculate half-maximal inhibitory concentrations (EC50 values). The most potently inhibitory mAbs (EC50: <10 ng/mL) belonged to groups A, B, and C, and also blocked ACE2 binding (FIG. 1). Some mAbs in group C and D that did not block ACE2 binding still showed robust neutralizing activity (EC50: 20-100 ng/mL), although the majority were weakly inhibitory. Group E mAbs were weakly neutralizing and did not block ACE2 binding.

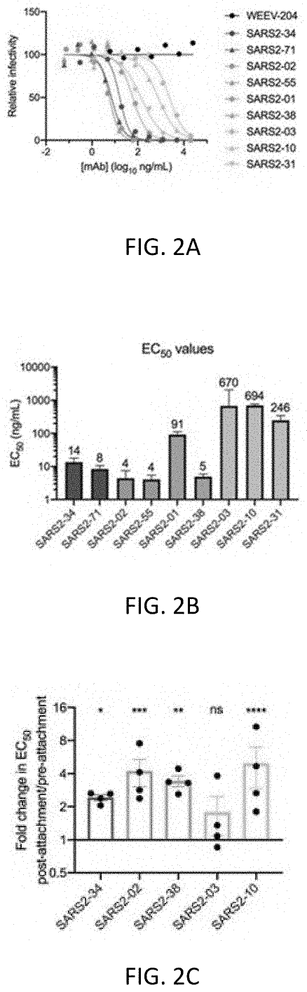

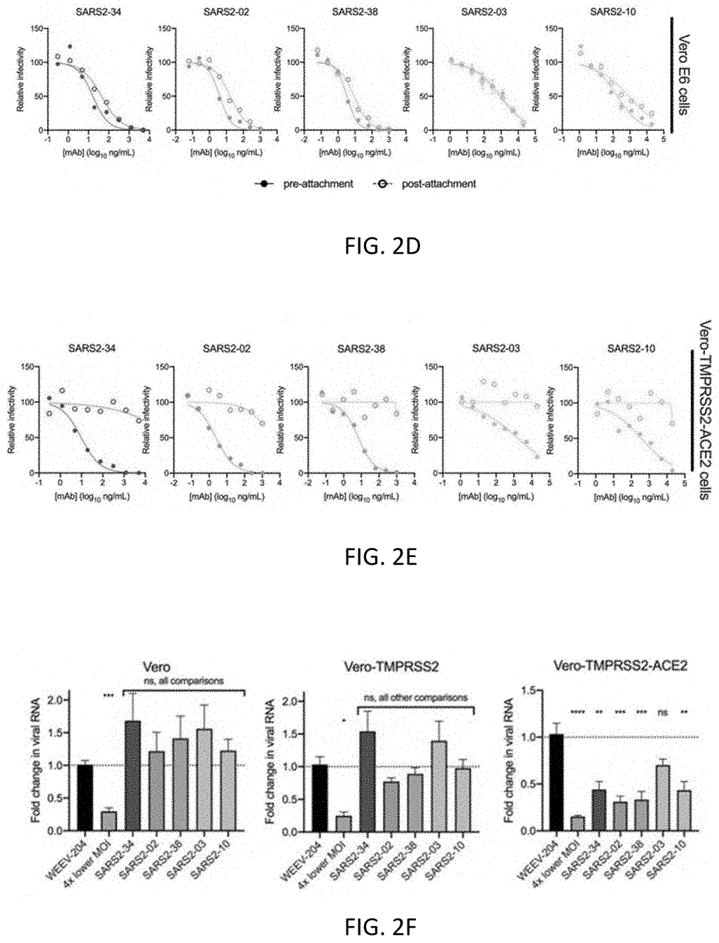

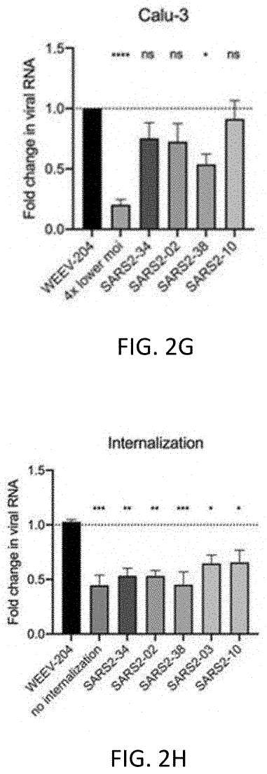

A subset of mAbs from groups A, B, C, and D were selected for more detailed study. two mAbs were chosen with the highest neutralization potency from each group; in cases where mAbs had high variable region sequence similarity, only one of these mAbs were selected for further study. SARS2-03 was also selected, as it was one of the few neutralizing mAbs that did not block ACE2 binding. Nine mAbs were purified from hybridoma supernatants and retested for neutralization potency by FRNT using Vero cells and the WA1/2020 isolate (FIG. 2A-2B). Again, the most potently neutralizing purified mAbs belonged to groups A, B, and C, with less inhibitory activity in those derived from group D. These nine mAbs were also characterized for competition binding with each other. The two group A mAbs (SARS2-34 and SARS2-71) competed for spike binding only with each other. In contrast, mAbs in groups B (SARS2-02 and SARS2-55) and C (SARS2-01 and SARS2-38) competed for spike binding across both groups. SARS2-03, a group D mAb, did not bind spike efficiently in the presence of group B or C mAbs and blocked binding of group C mAb SARS2-01. SARS2-10 and SARS2-31, the other two group D mAbs, however, competed with only each other. Together, these results suggest that mAbs in group C may have overlapping epitopes with group B mAbs and group D mAb SARS2-03, whereas group A mAbs and the remaining group D mAbs likely engage physically distinct epitopes.

Mechanism of neutralization by anti-SARS-CoV-2 mAbs: Whether the anti-SARS-CoV-2 mAbs inhibited infection at a pre- or post-attachment step of the entry process was investigated. For these experiments, one representative mAb from groups A, B, and C (SARS2-34, SARS2-02, and SARS2-38, respectively) and two mAbs from group D (one that blocks ACE2 binding, [SARS2-10] and one that does not [SARS2-03]) were selected. The neutralization potency of mAbs was compared when added before or after virus absorption to Vero E6 cells. Unexpectedly, all mAbs retained neutralizing activity when added post-attachment, although the potency of groups A, B, and C mAbs SARS2-02, SARS2-34, and SARS2-38 was reduced slightly (˜2- to 4-fold, p<0.05) relative to pre-attachment neutralization titers (FIG. 2C-2D). SARS2-10, a group E mAb, also showed a ˜5-fold decrease (p<0.0001) in neutralizing activity when added after attachment. In contrast, SARS2-03, another group E mAb, and the only mAb in this smaller panel that did not block ACE2-spike interactions, had similar neutralization potencies (p=0.79) when added before or after cell attachment. These data suggest that mAbs that inhibit spike protein binding to ACE2 neutralize SARS-CoV-2 slightly more efficiently when given at a pre-attachment step, although all of the mAbs tested retained the ability to inhibit infection when given after virus attachment to cells.

To determine the impact of entry factor expression on target cells on virus neutralization, these findings were extended to cells that ectopically express human ACE2 and TMPRSS2. In contrast to the relatively minor change in neutralization potency seen with all mAbs for pre- versus post-attachment observed using Vero E6 cells, mAbs no longer efficiently neutralized SARS-CoV-2 infection when added after attachment to Vero-TMPRSS2-ACE2 cells, although pre-attachment neutralization activity remained intact (FIG. 2E). Thus, the ability of anti-SARS-CoV-2 mAbs to neutralize at a post-attachment step depended on expression levels of viral entry factors and the cell line.

The ability of the mAbs to block directly virus attachment to cells was also tested, including Vero E6, Vero-TMPRSS2, and Vero-TMPRSS2-ACE2 cells. None of the mAbs efficiently blocked SARS-CoV-2 attachment to Vero or Vero-TMPRSS2 cells (FIG. 2F). However, with the exception of SARS2-03, all mAbs reduced virus attachment to Vero-TMPRSS2-ACE2. To corroborate these findings with cells that endogenously express human ACE2, experiments were repeated with Calu-3 cells, a human lung epithelial cell line. An intermediate phenotype with Calu-3 cells was observed, with modest attachment inhibition by mAbs in groups A, B, and C; levels of attached virus were ˜25-50% lower than the isotype mAb control with inhibition by only SARS2-38 attaining statistical significance (FIG. 2G). This result suggests that the anti-RBD mAbs can inhibit viral attachment to cells, but this activity depends on levels of human ACE2 expression. Since the mAbs did not efficiently inhibit attachment to Vero E6 cells lacking human ACE2 expression, it was tested whether they block a later step in the entry process by using a virus internalization assay. In Vero E6 cells, pre-incubation with all of the anti-RBD mAbs tested resulted in reduced levels of internalized virus (FIG. 2H).

Because we observed cell type-dependent differences in the mechanism of neutralization, the effect of cell substrate on the inhibitory potency of our anti-RBD mAbs was tested by FRNT. Notably, the anti-RBD mAbs neutralized SARS-CoV-2 WA1/2020 equivalently in Vero E6, Vero-TMRPSS2, and Vero-TMPRSS2-ACE2 cells. Thus, although the mAbs variably block SARS-CoV-2 attachment on different cell types, the potency of infection inhibition was similar across cell substrates. This result may be explained by the ability of anti-RBD mAbs to block a required ACE2-dependent entry interaction in all of the cell substrates tested, even though the attachment step is variably affected.

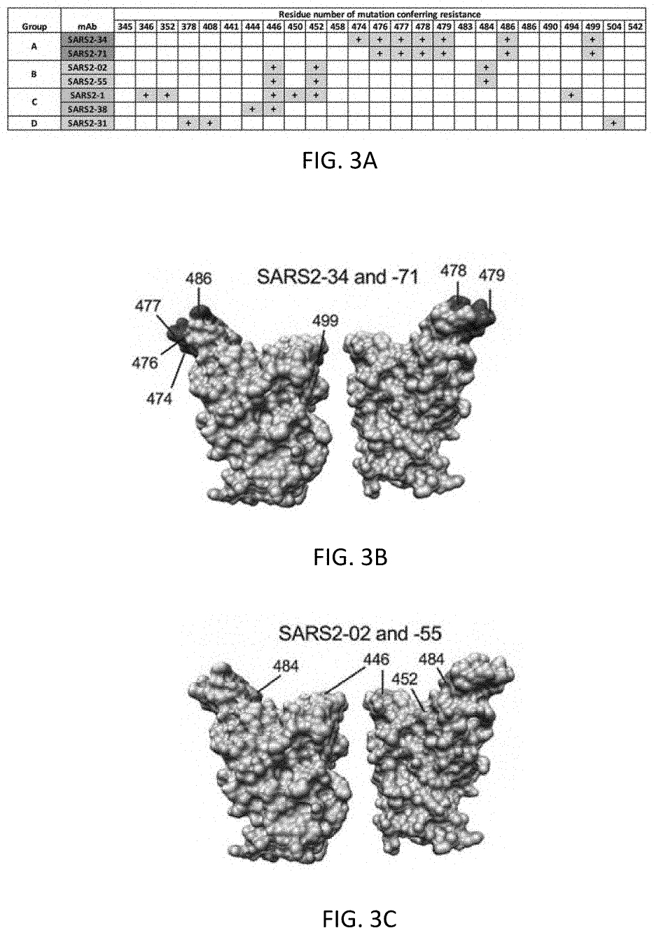

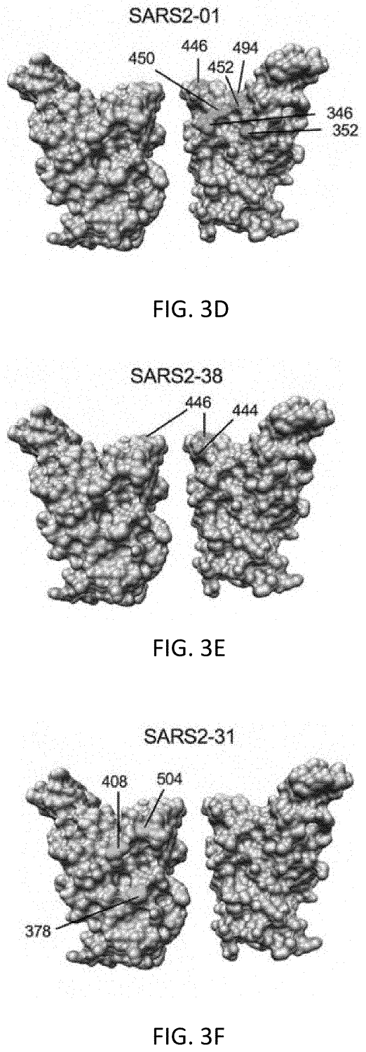

Epitope mapping of anti-SARS-CoV-2 mAbs using neutralization escape analysis: To determine spike residues important for recognition by anti-SARS-CoV-2 mAbs, neutralization escape mutants were previously isolated by passaging a VSV-eGFP-SARS-CoV-2-S chimeric virus in the presence of neutralizing mAbs. The above described subset of nine mAbs from groups A-D were tested for neutralization against the panel of sequenced mutants, with the exception of SARS2-10 and SARS2-03, which were not evaluated because of difficultly in isolating escape mutants with mAbs of low neutralization potency. Neutralizing activity was lost for group A mAbs SARS2-34 and SARS2-71 when residues 476-479, 486, and 499 were mutated; for group B mAbs SARS2-02 and SARS2-55 when residues 446, 452, and 484 were mutated; for group C mAb SARS2-01 when residues 346, 352, 446, 450, and 494 were mutated; for group C mAb SARS2-38 when residues 444 and 446 were mutated; and for group D mAb SARS2-31 when residues 378, 408, and 504 were mutated (FIG. 3A-3F).

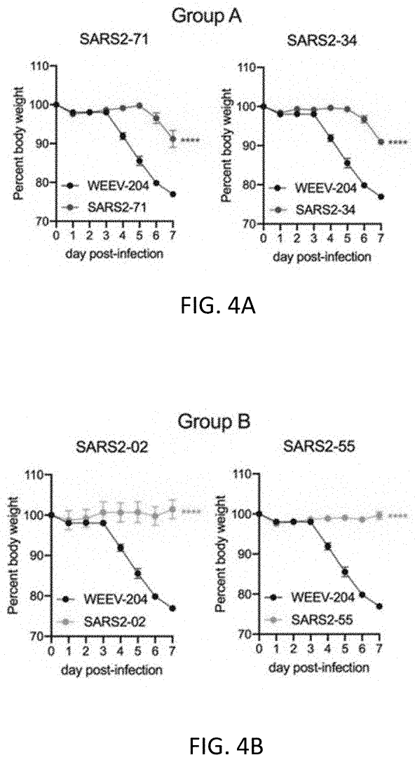

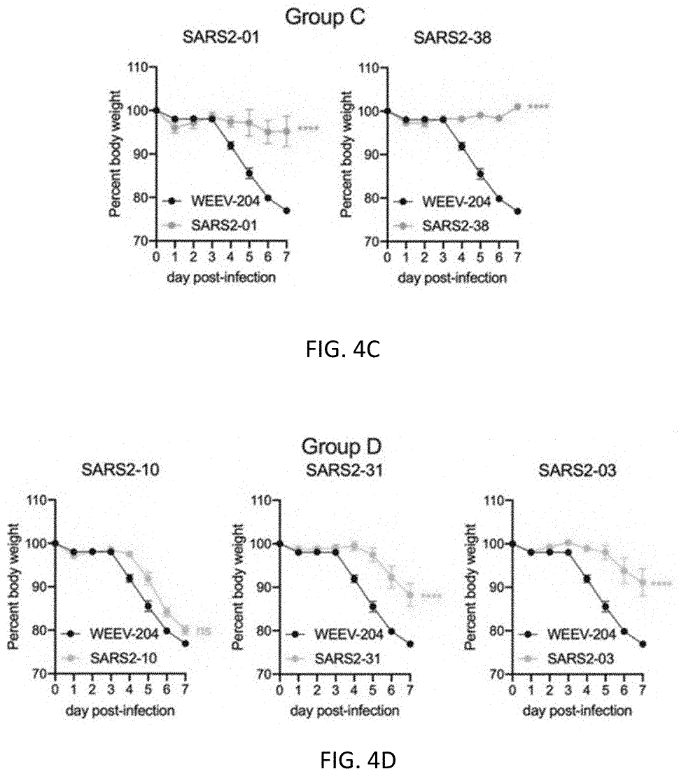

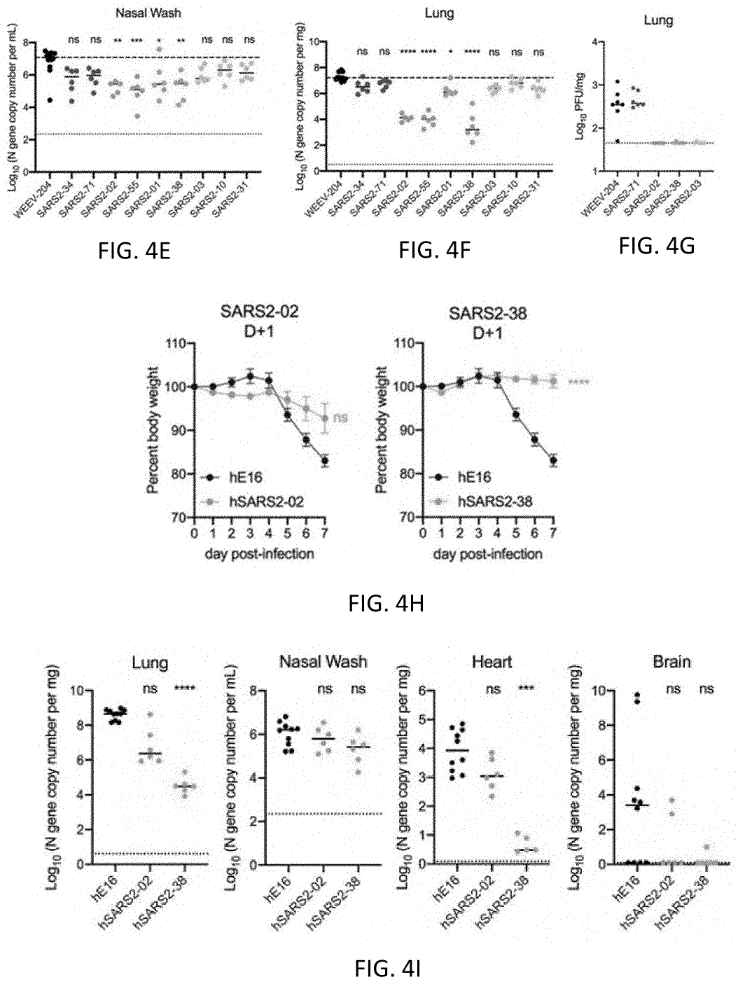

Anti-SARS-CoV-2 mAbs protect against virus challenge in vivo: Next the anti-SARS-CoV-2 mAbs were tested for protection against the historical SARS-CoV-2 WA1/2020 virus in vivo. Eight- to ten-week-old K18 human ACE2 (hACE2) transgenic mice were administered a single 100 μg dose (˜5 mg/kg) of anti-SARS-CoV-2 mAb 24 h prior to intranasal inoculation with 10³ FFU of SARS-CoV-2 WA1/2020. Mice treated with the isotype control mAb lost up to 25% body weight by 7 days post-infection (dpi), the designated endpoint of the study (FIG. 4A). Mice treated with group A mAbs SARS2-71 and SARS2-34 maintained body weight until 6-7 dpi, at which point a 10% weight loss was observed (FIG. 4A). Mice treated with group B mAbs SARS2-02 and SARS2-55 and group C mAbs SARS2-01 and SARS2-38 all maintained body weight throughout the experiment (FIG. 4B-4C). Animals treated with group D mAbs SARS2-10, SARS2-31 or SARS2-03 generally were less protected against virus-induced weight loss (FIG. 4D).

To corroborate these findings, the effect of mAb treatment on viral burden in the nasal washes and lungs on 7 dpi was measured. The greatest decreases in viral RNA levels (˜30 to 100-fold) in the nasal washes relative to isotype control mAb-treated mice were observed in animals treated with mAbs in group B (SARS2-02 and SARS2-55) and group C (SARS2-01 and SARS2-38) (FIG. 4E). The largest reductions in viral RNA levels in the lung (˜100 to 1,000-fold) again were observed for mice treated with mAbs in group B (SARS2-02 and SARS2-55) and group C (SARS2-38) (FIG. 4F). A smaller (˜10-fold) decrement of virus RNA levels in the lung was observed for group C mAb SARS2-03. The effects on infectious viral load were also measured in the lung by plaque assay for a subset of representative mAbs from each group. Whereas group A mAb SARS2-71 did not decrease the number of plaque-forming units (PFU) in the lung relative to the isotype control mAb-treated mice, SARS2-02, SARS2-38, and SARS2-03 all reduced infectious virus levels in the lung to the limit of detection of the assay (FIG. 4G). The lack of protection conferred by SARS2-71 in vivo was unanticipated given its potent neutralizing activity in cell culture (EC50 of 8 ng/mL, FIG. 2). Sequencing of viral RNA from the lungs of SARS2-71-treated mice at 7 dpi revealed an S477N mutation in the RBD in all samples, which was not present in the input WA1/2020 virus. Notably, S477N also emerged in vitro as an escape mutant under SARS2-71 selection pressure using the VSV-eGFP-SARS-CoV-2-S virus (FIG. 3A). Thus, despite its potent inhibitory activity in vitro, SARS2-71 likely failed to protect in vivo because of rapid emergence of a fully pathogenic escape mutant.

To evaluate further the level of protection conferred by a subset of mAbs in our panel, levels of cytokines and chemokines were measured in lung tissues at 7 dpi, which are markers of the inflammatory and pathological outcomes in this mouse model. SARS2-38 and SARS2-02 treatment resulted in substantially reduced cytokine and chemokine levels relative to isotype control mAb-treated mice, with levels equivalent to those seen in naïve mice. In contrast, treatment with SARS2-71 and SARS2-03 did not result in these reductions, with cytokine and chemokine levels similar to isotype-control treated and infected mice.

To test for post-exposure therapeutic protection against SARS-CoV-2 challenge, the variable regions of group B mAb SARS2-02 and group C mAb SARS2-38 were cloned and inserted into a human IgG1 backbone to make chimeric antibodies. Chimeric, humanized, or fully-human mAbs are more likely to be used in humans, and because Fc effector functions contribute to the therapeutic activity of neutralizing SARS-CoV-2 mAbs in vivo; the original murine IgG1 isotype of these mAbs binds poorly to the activating murine FcyRI and FcyRIV, whereas human IgG1 binds these murine Fc receptors with higher affinity and thus could have enhanced effector function. the neutralizing activity of the chimeric mAbs hSARS2-02 and hSARS-38 relative to the original murine IgG1 versions of the mAbs was confirmed. Next, K18-hACE2 mice were inoculated with 10³ FFU of SARS-CoV-2 WA1/2020. Twenty-four hours later, a single 200 μg (10 mg/kg) dose of hSARS2-02, hSARS2-38, or an isotype control mAb were administered. Both hSARS2-02 and hSARS2-38 protected against weight loss following infection (FIG. 4H). At 7 dpi, hSARS2-38 reduced viral RNA levels in the lung and heart by ˜10,000-fold, whereas hSARS2-02 reduced infection by only ˜10-100 fold in these tissues (FIG. 4I).

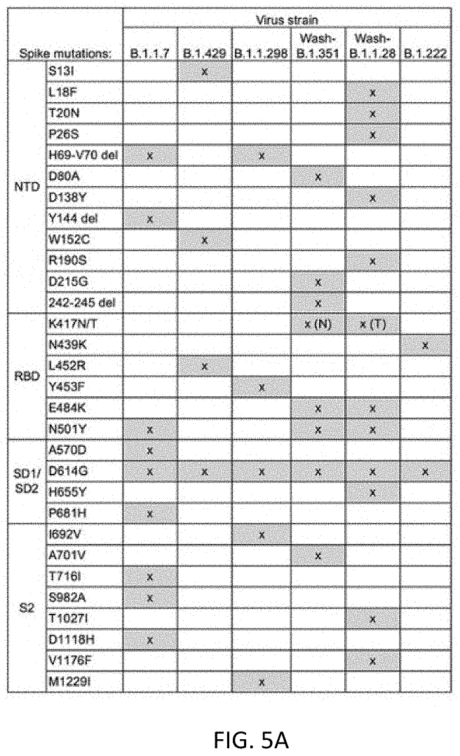

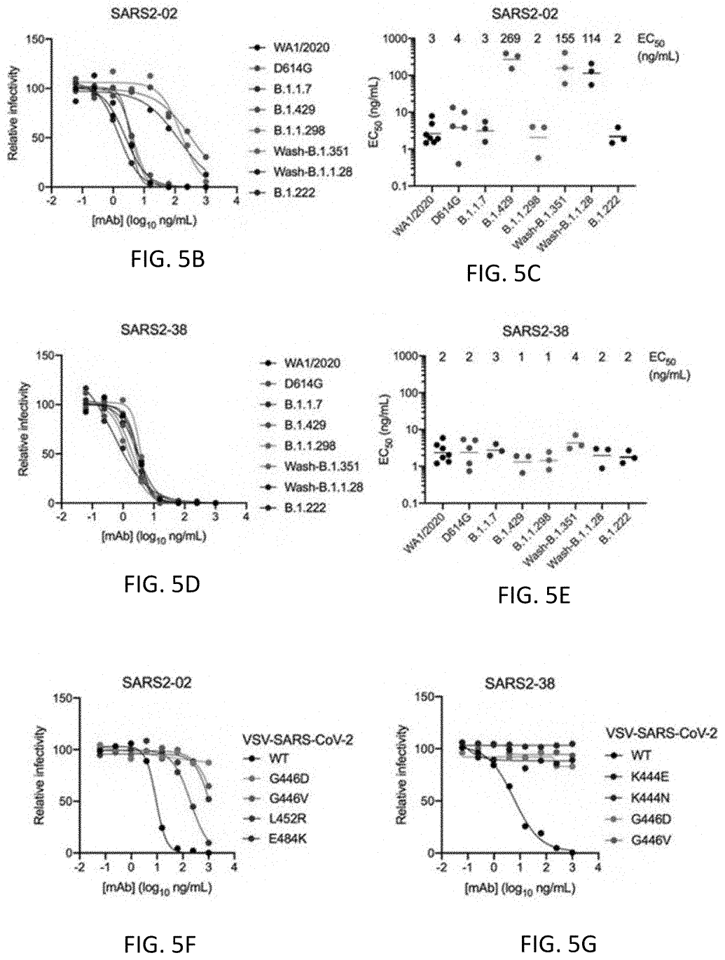

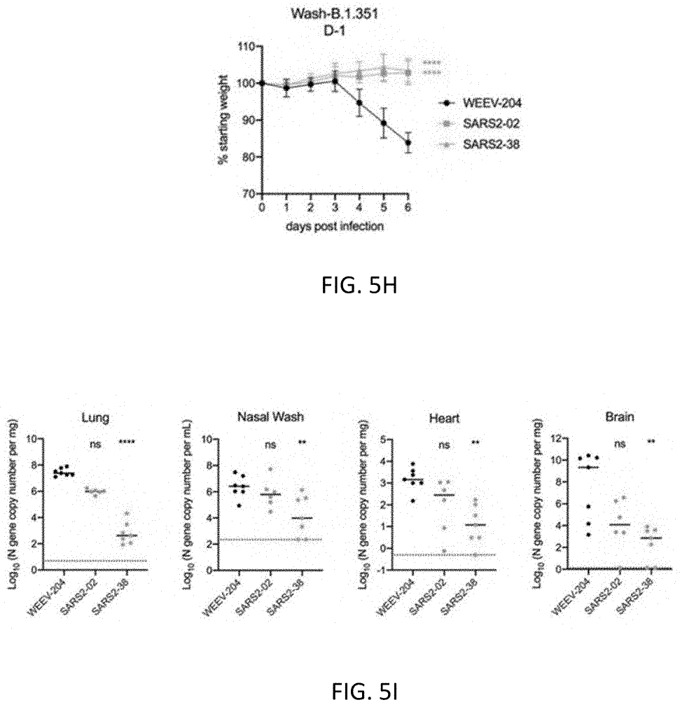

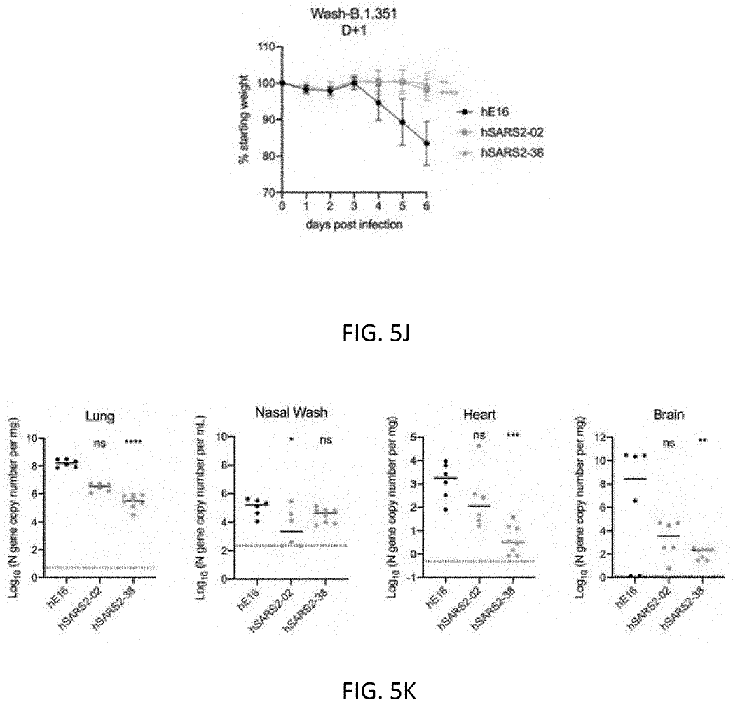

Neutralization of variants of concern by anti-SARS-CoV-2 mAbs: The two mAbs (SARS2-02 and SARS2-38) that conferred the greatest protection against WA1/2020 were tested in vivo for neutralization of viruses with spike proteins corresponding to circulating variants of concern (VOCs). Recombinant chimeric WA1/2020 viruses encoding the spike protein from B.1.351 or B.1.1.28 were utilized for these studies (Wash-1.351 and Wash-1.1.28), as well as WA1/2020 with an introduced D614G mutation; viral isolates B.1.1.7, B.1.429, B.1.1.298, and B.1.222 were also tested. Several of these VOCs encode amino acid changes in spike that can affect mAb binding (FIG. 5A), including changes identified in our VSV-eGFP-SARS-CoV-2-S escape mutant panel: L452R and E484K both showed reduced sensitivity to neutralization of VSV-eGFP-SARS-CoV-2-S by group B mAbs SARS2-02 and SARS2-55. Indeed, SARS2-02 exhibited reduced (˜50-100-fold) neutralizing activity against authentic SARS-CoV-2 strains with E484K (Wash-B.1.351 and Wash-B.1.1.28) or L452R (B.1.429) substitutions (FIG. 5B and FIG. 5C). Notably, SARS2-38 did not lose potency against any of the variant viruses, with EC50 values ranging from 1-4 ng/mL across the panel tested (FIG. 5D and FIG. 5E).

To expand on this analysis, the VSV-eGFP-SARS-CoV-2-S viruses that were resistant to SARS2-02 and SARS2-38 were tested for neutralization using full dose response curves analysis. SARS2-02 showed ˜20-fold reduced potency against E484K, ˜100-fold reduced potency against L452R and G446V, and did not neutralize G446D at the highest concentration of mAb tested (FIG. 5F). SARS2-38 showed virtually no neutralizing activity against K444E, K444N, G446D, or G446V mutants even at the highest concentration (1 μg/ml) of mAb tested (FIG. 5G). Despite these results with VSV-eGFP-SARS-CoV-2-S viruses, when authentic WA1/2020 D614G or Wash-B.1.351 SARS-CoV-2 in Vero-TMPRSS2-ACE2 cells were serially passaged in the presence of neutralizing mAbs, resistant viruses were readily isolated following SARS-02 but not SARS2-38 selection with both strains.

SARS2-02 and SARS2-38 were tested for protection against Wash-B.1.351 in K18-hACE2 mice. Animals treated with 100 μg of either SARS2-02 or SARS2-38 24 h prior to infection were protected from weight loss (FIG. 5H), despite the reduced neutralization potency of SARS2-02 against Wash B.1.351. SARS2-38 treatment greatly reduced viral titers in the lung, nasal washes, heart, and brain at 7 dpi compared to the isotype control-treated mice, whereas SARS2-02 had less of a protective effect (FIG. 5I). When hSARS2-02 and hSARS2-38 were administered to the K18-hACE2 transgenic mice as therapy 24 h after infection with Wash-B.1.351, a similar phenotype was observed: while they both protected mice against weight loss (FIG. 5J), hSARS2-38 resulted in a greater reduction in viral titers at 7 dpi in the lung, heart, and brain than hSARS2-02 (FIG. 5K).

SARS2-38 targets the proximal RBM ridge with extensive light chain contact: To define further the mechanistic basis for the broad and potent neutralization by SARS2-38, the interaction of antigen binding fragments (Fab) of SARS2-38 with SARS-CoV-2 spike using biolayer interferometry (BLI) was first analyzed. SARS2-38 bound spike with high monovalent affinity (kinetically derived KD of 6.5 nM) and had a half-life of 4.8 min. To understand the basis for this binding structurally, cryo-electron microscopy (cryo-EM) on complexes of SARS2-38 Fab and the SARS-CoV-2 spike protein was performed. three-dimensional classes to sample the conformational landscape of the Fab/spike complex were generated, and the class of highest resolution was refined further. This class consisted of trimeric spike with all RBDs in the down position (D/D/D) and one RBD bound by Fab (FIG. 6A). Using non-uniform refinement, an overall resolution of 3.20 Å was achieved, with local resolution ranging from ˜2.5 Å in the core of the spike to ˜5.5 Å in the constant region of the Fab, which was visible only at high contour. Other binding configurations also were seen, the most predominant consisting of spike with one RBD up and two RBDs down (U/D/D), with only the up RBD bound by Fab (31.1%). Less frequently, all three RBDs were bound by Fab in the U/D/D conformation (22.0%). Although SARS2-38 could bind SARS-CoV-2 spike with full occupancy, 61.1% of spike trimers were bound only by a single Fab molecule.

To improve resolution at the Fab/RBD interface in the D/D/D reconstruction, a focused, local refinement of the SARS2-38 variable domain (Fv) and RBD was performed, excluding the rest of the spike and the constant region of the Fab. This reconstruction of the Fv/RBD complex achieved a resolution of 3.16 Å, allowing unambiguous placement of the protein backbone, secondary structures, and most side chains at the interface. The SARS2-38 Fv sits atop three loops protruding at the proximal end of the RBM between helix α1 and strand β1 (contact residues T345-R346), strands β4 and β5 (N439-G446, N448-Y451), and strand β6 and helix α5 (S494 and Q498-T500; FIG. 6A-6B); these results correspond well with our VSV-based escape mutant mapping (FIG. 3). All three light chain CDRs contact loop β4-β5, with CDR2 and CDR3 forming additional contacts with loops α1-β1 and β6-α5, respectively. In comparison, the heavy chain interacts in a more limited manner with loops β4-β5 and β6-α5 via CDR2 and CDR3. CDR1 of the heavy chain makes no contact at all with the RBD. The heavy chain does, however, engage ACE2 contact residues of the RBM (FIG. 6A, right panel). This and other steric effects likely explain the inhibition of ACE2 binding by SARS2-38.

The SARS2-38 epitope is conserved among circulating SARS-CoV-2 variants of concern: SARS2-38 potently neutralized all tested VOCs. To understand this broadly-neutralizing activity, the SARS2-38 epitope was mapped alongside VOC mutations within the RBD (FIG. 6B, left panel and FIG. 6C). One mutation in the SARS2-38 footprint, N439K, is present in variant B.1.222 and resides at the periphery of the epitope. However, B.1.222 remained sensitive to neutralization by SARS2-38, and escape mutants at this residue were not generated in vitro, suggesting that N439 is not critical for SARS2-38 binding. The SARS2-38 epitope includes no other residues corresponding to VOC mutations, which explains its performance against these variants. Notwithstanding this, we could select escape mutations in vitro in the context of VSV-eGFP-SARS-CoV-2-S chimeric virus, namely K444E/N and G446DN substitutions, which reside on the β4-β5 loop central to the SARS2-38 epitope (FIG. 6B-C). The substitutions generated at K444 result in a loss of positive charge (K444N) or charge reversal (K444E), whereas the mutation at G446 may distort the entire loop structure; in our model, G446 adopts a stereochemistry unique to the glycine residue (φ=108° and =ψ−19°). This structural analysis likely explains the resistance conferred by these amino acid substitutions.

To understand the efficacy of SARS2-38 amidst the landscape of all circulating variants, the COVID-19 CoV Genetics Browser was used (covidcg.org) to probe RBD sequences in the GISAID database (786,273 isolates as of Mar. 28, 2021). a log-scale conservation score for RBD residues 333-520 was then developed. In this model, perfect conservation of the reference amino acid (from 2019n-CoV/WA1/2020) across all isolates corresponds to a score of 1, and complete loss of the reference amino acid results in a score of 0. Visualizing these scores on a color-coded RBD surface rendering (blue=1, more conserved; red=0, more variable) revealed that the RBM is generally more variable than the rest of the RBD, with VOCs clearly seen as red patches (FIG. 6B, right panel). This analysis also suggested that in addition to not being affected by the VOCs tested in this study, SARS2-38 targets a portion of the RBM that is conserved among circulating SARS-CoV-2 variants. The positions at which escape mutants were identified using VSV-eGFP-SARS-CoV-2-S chimeric viruses were substituted in only 0.02% (K444) and 0.04% (G444) of isolates, with the specific escape mutations (K444E/N and G446D/V) observed in only 0.007% and 0.03% of isolates respectively. Overall, 99.96% of isolates lacked the escape mutations for SARS2-38 identified in our study.

### DISCUSSION

In this example, a panel of mAbs that bind the RBD of the SARS-CoV-2 spike protein were described and characterized extensively. Several anti-RBD mAbs protected in vivo against SARS-CoV-2 infection in K18-hACE2 transgenic mice. While the less potently neutralizing mAbs directed against epitopes on the base of RBD (SARS2-10, SARS2-31, and SARS2-03) exhibited diminished protection against weight loss, induction of inflammatory cytokines and chemokines in the lung, and viral infection in the lung and nasal wash than mAbs recognizing the RBM, neutralization potency was not the only predictor of in vivo efficacy. Indeed, SARS2-71 neutralized SARS-CoV-2 with a potency similar to that of protective mAbs SARS2-02 and SARS2-38, yet failed to confer protection in mice. Notwithstanding this result, antibodies targeting proximal competing epitopes as SARS2-71, such as COV2-2196, have been shown to confer protection in vivo. The failure of SARS2-71 to protect in particular is likely due to the emergence of the escape variant S477N in vivo. This finding demonstrates that SARS-CoV-2 can rapidly escape from mAb inhibition in vivo, and that mAb or mAb cocktails that prevent or limit rapid escape mutant generation likely will have greater therapeutic utility. While currently authorized mAb treatments include cocktails, the emergence of VOCs that are resistant to one or both component mAbs could compromise drug efficacy.

The most potently inhibitory mAbs in our panel bind epitopes within or proximal to the RBM and inhibit spike interaction with human ACE2 by ELISA, as observed for other anti-SARS-CoV-2 mAbs. Several of these mAbs inhibited viral attachment to Calu-3 and Vero-TMPRSS2-ACE2 cells, but not to Vero E6 cells or Vero-TMPRSS2 cells. Infection of Vero E6 cells by SARS-CoV-2 is dependent on endogenous levels of monkey ACE2 expression, as pretreatment with anti-ACE2 mAbs inhibits infection. However, other host factors such as heparan sulfate also can mediate virus attachment to cells. If binding to other cell surface ligands occurs prior to the RBD-ACE2 interaction, mAbs that block ACE2 binding may not efficiently inhibit SARS-CoV-2 attachment, but instead block a downstream ACE2-dependent entry step. This idea is supported by our data showing that several neutralizing mAbs block viral internalization in Vero E6 cells. Moreover, anti-RBD mAbs have only moderate decreases in neutralization potency when added after virus absorption to Vero E6 cells. In contrast, when SARS-CoV-2 attaches to the cell surface via human ACE2 interaction, such as in Vero-TMPRSS2-ACE2 cells, the addition of anti-RBD mAbs after attachment failed to neutralize virus infection. A higher density of ACE2 or higher affinity of spike protein for human ACE2 (relative to monkey ACE2) on the Vero-TMPRSS2-ACE2 cells may drive initial virus attachment through the RBD-ACE2 interaction and explain why mAbs can block this step in these cells. Together, these data suggest that the ability of anti-RBD mAbs to inhibit SARS-CoV-2 attachment depends on cellular ACE2 expression levels and thus can be cell-type dependent. As these mechanistic differences did not markedly affect mAb potency on the different cellular substrates, it was concluded that in the cells tested there is a required entry interaction with ACE2 either at attachment, post-attachment, or internalization steps.

Several mutations and deletions in emerging VOCs occur in the NTD and RBD that allow them to avoid antibody recognition, including RBD mutations K417N/T (B.1.351 and B.1.1.28), N439K (B.1.222), L452R (B.1.429), Y453R (B.1.1.298), E484K (B.1.351 and B.1.1.28), and N501Y (B.1.1.7, B.1.351, and B.1.1.28), highlighting the importance of developing mAbs against a variety of spatially distinct epitopes. In our panel, SARS2-38 potently neutralized viruses encoding any of the above mutations, did not readily select for escape mutations with authentic SARS-CoV-2 strains, and retained therapeutic activity in vivo against a virus containing substitutions of one of the key VOCs (B.1.351). Moreover, functional mapping and structural analysis of the binding footprint of SARS-CoV-2 defined a conserved RBD epitope that could be recognized by other potently neutralizing and protective human mAbs.

Relatively few antibodies targeting similar epitopes to SARS2-38 have been described, and those characterized bind the RBD in distinct orientations with heavy chain predominance (FIG. 7A). These include murine mAb 2H04, as well as human mAbs REGN10987, COV2-2130, and, though less similar, S309. SARS2-38 differs in two respects: (a) the baseline neutralizing activity of SARS2-38 against WA1/2020 in Vero cells (EC50, ˜5 ng/mL) is 30-fold, 20-fold, and 16-fold more potent than that of 2H04, COV2-2130, and S309, respectively; and (b) SARS2-38 retains strong neutralization potency against all VOCs evaluated in this example, whereas the inhibitory activity 2H04, COV2-2130, and S309 is reduced somewhat against B.1.1.7, B.1.429, and B.1.351, respectively. Similarly, REGN10987 exhibited a 10-fold reduction in neutralizing activity against B.1.429 compared to WA1/2020. A structural examination of these other antibody footprints within the context of VOC mutations does not provide a direct explanation for some of the resistance (FIG. 7B). Instead, allostery may play a role. Whereas other broadly and potently neutralizing mAbs (including mAbs 2C08, COV2-2196, 58G6, 510A5, and 52X259) have been reported that bind RBD epitopes at residues G476, F486, and N487, or loops near residues 369-386, 404-411, 450-458, and 499-508, SARS2-38 targets a distinct epitope proximal to the RBM and has been evaluated functionally against a larger panel of authentic viruses containing sequences corresponding to emerging SARS-CoV-2 variants.

In summary, the present example characterized a panel of anti-SARS-CoV-2 mAbs, defined their cellular mechanism of action in different cells, tested in vitro neutralizing and in vivo protection capacity against historical and circulating variants, and determined the structure of the viral spike protein bound to SARS2-38, a potently and broadly neutralizing mAb that recognizes emerging VOCs. A humanized version of SARS2-38 confers therapeutic protection against the WA1/2020 isolate and a SARS-CoV-2 strain expressing the spike protein of B.1.351. The conserved epitope bound by SARS2-38 thus may be a potential target for antibodies with therapeutic potential or that are induced by effective vaccines with more limited potential for resistance against VOCs.

### MATERIALS AND METHODS

Viruses: The 2019n-CoV/USA_WA1/2020 (WA1/2020) isolate of SARS-CoV-2 was obtained from the US Centers for Disease Control (CDC). WA1/2020 stocks were propagated on Vero CCL81 cells and used at passage 6 and 7. Viral titer was determined by focus-forming assay (FFA) on Vero E6 cells as described (Case et al., 2020). The D614G virus was produced by introducing the mutation into an infectious clone of WA1/2020, and the B.1.351 and B.1.1.28 Spike genes were cloned into the WA1/2020 infectious clone to produce Wash-B.1.351 and Wash-B.1.1.28 chimeric viruses, as described previously. The B.1.1.7, B.1.429, B.1.298, and B.1.222 isolates were isolated from infected individuals. Viruses were propagated on Vero-TMPRSS2 cells and subjected to deep sequencing to confirm the presence of the substitutions indicated in FIG. 5A.

Cells: Cell lines were maintained at 37° C. in the presence of 5% CO2. Vero E6 cells were passaged in Dulbecco's Modified Eagle Medium (DMEM) (Invitrogen) supplemented with 10% fetal bovine serum (FBS) (Omega Scientific) and 100 U/mL penicillin-streptomycin (P/S) (Invitrogen). Vero cells that over-express TMPRSS2 or TMPRSS2-ACE2 were maintained as Vero CCL81 cells, with the addition of 5 μg/mL blasticidin (Vero-TMPRSS2) or 10 μg/mL puromycin (Vero-TMPRSS2-ACE2). Calu-3 cells were maintained in DMEM with 20% FBS and 100 U/mL P/S.

Proteins: Genes encoding SARS-CoV-2 spike protein (residues 1-1213, GenBank: MN908947.3) and RBD (residues 319-514) were cloned into a pCAGGS mammalian expression vector with a C-terminal hexahistidine tag. The spike protein was prefusion stabilized and expression optimized via six proline substitutions (F817P, A892P, A899P, A942P, K986P, V987P), with a disrupted S1/S2 furin cleavage site and a C-terminal foldon trimerization motif (YIPEAPRDGQAYVRKDGEWVLLSTFL). Expi293F cells were transiently transfected, and proteins were recovered via cobalt-charged resin chromatography (G-Biosciences) as previously described. For ACE2 binding inhibition analysis, the SARS-CoV-2 spike protein was made by synthesizing a gene encoding the ectodomain of a prefusion conformation-stabilized SARS-CoV-2 spike (S6Pecto) protein containing C-terminal Twin-Strep-tag. The spike gene was then cloned it into a DNA plasmid expression vector for mammalian cells. Protein was produced in FreeStyle 293-F cells (Thermo Fisher Scientific) and purified from culture supernatants using StrepTrap HP affinity column (Cytiva).

Mice: Animal studies were carried out in accordance with the recommendations in the Guide for the Care and Use of Laboratory Animals of the National Institutes of Health. The protocols were approved by the Institutional Animal Care and Use Committee at the Washington University School of Medicine (Assurance number A3381-01). Virus inoculations were performed under anesthesia that was induced and maintained with ketamine hydrochloride and xylazine, and all efforts were made to minimize animal suffering.

K18-hACE2 transgenic mice were purchased from Jackson Laboratories (#034860) and housed in a pathogen-free animal facility at Washington University in St. Louis. For passive transfer studies, mAbs were diluted in PBS and administered to mice via intraperitoneal injection in a 100 μL total volume. Viral infections were performed via intranasal inoculation with 103 FFU of virus. Mice were monitored daily for weight loss.

MAb generation: BALB/c mice were immunized with 10 μg of SARS-CoV-2 RBD adjuvanted with 50% AddaVax™ (InvivoGen), via intramuscular route (i.m.), followed by i.m. immunization two and four weeks later with SARS-CoV-2 spike protein (5 μg and 10 μg, respectively) supplemented with AddaVax™. Mice received a final, non-adjuvanted boost of 25 μg of SARS-CoV-2 spike or RBD (12.5 μg intravenously and 12.5 μg interperitoneally) 3 days prior to fusion of splenocytes with P3X63.Ag.6.5.3 myeloma cells. Hybridomas producing antibodies that bound to SARS-CoV-2-infected permeabilized Vero CCL81 cells by flow cytometry and to SARS-CoV-2 recombinant spike protein by direct ELISA were cloned by limiting dilution. All hybridomas were screened initially with a single-endpoint neutralization assay using hybridoma supernatant diluted 1:3 and incubated with SARS-CoV-2 for 1 h at 37° C. prior to addition to Vero E6 cells. Following a 30-h incubation, cells were fixed, permeabilized, and stained for SARS-CoV-2 infection with CR3022 as described previoulsy. A subset of neutralizing hybridoma supernatants were purified commercially (Bio-X Cell) after adaptation for growth under serum-free conditions.

VSV-eGFP-SARS-CoV-2-S escape mutants: VSV-eGFP-SARS-CoV-2-S escape mutants were produced as described previously. Briefly, plaque assays were performed to isolate escape mutants on Vero-TMPRSS2 cells with neutralizing mAb in the overlay. Escape clones were plaque-purified on Vero-TMPRSS2 cells in the presence of mAb. Plaques in agarose plugs and viral stocks were amplified on MA104 cells at an MOI of 0.01 in Medium 199 containing 2% FBS and 20 mM HEPES pH 7.7 (Millipore Sigma) at 34° C. Viral supernatants were harvested upon extensive cytopathic effect and clarified of cell debris by centrifugation at 1,000×g for 5 min.

Determination of mAb concentration in hybridoma supernatant: The mAb concentration in each hybridoma supernatant was quantified by ELISA. Nunc MaxiSorp plates (Thermo Fisher Scientific) were coated with 1 μg/mL of goat anti-mouse IgG (Southern Biotech) in 50 μL of NaHCO3 (pH 9.6) coating buffer and incubated overnight at 4° C. Plates were washed three times with ELISA wash buffer (PBS containing 0.05% Tween-20), and then incubated with 200 μL of blocking buffer (PBS, 2% BSA, 0.05% Tween-20) for 1 h at room temperature. Plates were incubated with hybridoma supernatant diluted 1:500 or 1:2000 in blocking buffer, or serial dilutions of purified isotype control mAb as a standard, for 1 h at room temperature. Plates were washed three times with ELISA wash buffer, and incubated with 50 μL of anti-mouse IgG-HRP (Sigma) diluted 1:500 for 1 h at room temperature. Plates were washed three times with ELISA wash buffer and three times with PBS, before incubation with 100 μL of TMB substrate (Thermo Fisher Scientific) for 3 min at room temperature before quenching with the addition of 50 μL of 2 N H₂SO₄ and measuring OD 450 nm. Antibody concentrations in hybridoma supernatant were interpolated from a standard curve produced using an isotype control mAb.

Spike and RBD binding analysis: 96-well Maxisorp plates were coated with 2 μg/mL of SARS-CoV-2 spike or RBD protein in 50 mM Na2CO3 (70 μL) overnight at 4° C. Plates were washed three times with PBS+0.05% Tween-20 and blocked with 200 μL of PBS+0.05% Tween-20+1% BSA+0.02% NaN3 for 2 h at room temperature. 75 μL of blocking buffer and 50 μL of hybridoma supernatant were combined, and 50 μL/well of diluted supernatants were added to the plates and incubated for 1 h at room temperature. Bound IgG was detected using HRP-conjugated goat anti-mouse IgG (at 1:2,000). Following a 1 h incubation, washed plates were developed with 50 μL of 1-Step Ultra TMB-ELISA, quenched with 2 N H₂SO₄, and the absorbance was read at 450 nm.

Competition binding analysis: The assay was performed as described previously. Briefly, for screening study wells of 384-well microtiter plates were coated with 1 g/mL of purified SARS-CoV-2 S6Pecto protein at 4° C. overnight. Plates were blocked with 2% bovine serum albumin (BSA) in DPBS-T for 1 h. Mouse hybridoma culture supernatants were diluted five-fold in blocking buffer, added to the wells (20 μl per well) in duplicates for each tested reference mAb and incubated for 1 h at room temperature. Biotinylated reference human mAbs with known epitope specificity (COV2-2130, COV2-2196, and CR3022 were added to each of well with the respective hybridoma culture supernatant at 1.25 μg/mL in a volume of 5 μl per well (final concentration of biotinylated mAb, 0.25 μg/mL) without washing of the plates, and then incubated for 1 h at room temperature. Plates then were washed, and bound antibodies were detected using HRP-conjugated avidin (Sigma, A3151, 0.3 μg/mL final concentration) and a TMB substrate. The signal obtained for binding of the biotin-labelled reference antibody in the presence of the hybridoma culture supernatant was expressed as a percentage of the binding of the reference antibody alone after subtracting the background signal. Tested mAbs were considered competing if their presence reduced the reference antibody binding to less than 41% of its maximal binding and non-competing if the signal was greater than 71%. A level of 40-70% was considered intermediate competition.

Human ACE2 binding inhibition analysis: The assay was performed as described previously. Briefly, for screening study wells of 384-well microtiter plates were coated with 1 μg/mL purified recombinant SARS-CoV-2 S6Pecto protein at 4° C. overnight. Plates were blocked with 2% non-fat dry milk and 2% normal goat serum in DPBS-T for 1 h. Mouse hybridoma culture supernatants were diluted five-fold in blocking buffer, added to the wells (20 μl per well) in quadruplicate, and incubated for 1 h at room temperature. Recombinant human ACE2 with a C-terminal Flag tag peptide was added to wells at 2 μg/mL in a 5 μl per well volume (final 0.4 μg/mL concentration of human ACE2) without washing of the plates, and then incubated for 40 min at room temperature. Plates were washed and bound human ACE2 was detected using HRP-conjugated anti-Flag antibody (Sigma-Aldrich, A8592, 1:5,000 dilution) and TMB substrate. ACE2 binding without antibody served as a control for maximal binding. Antibody COV2-2196 (RBD) served as a control for ACE2 binding inhibition. The signal obtained for binding of the human ACE2 in the presence of each dilution of tested culture supernatant was expressed as a percentage of the human ACE2 binding without antibody after subtracting the background signal.

Sequencing, cloning, and expression of chimeric IgG1: To generate chimeric human IgG1 from mouse hybridoma cell lines, cells were lysed in Trizol (Thermo) followed by RNA purification with Direct-Zol Micro kit (Zymo). 5′ RACE products were generated with Template Switching RT Enzyme Mix (New England Biolabs) using anchored poly(dT)23 and TSO oligonucleotides according to the manufactures instructions. Heavy and light chain sequences were amplified with primers specific for the TSO handle-sequence and the respective constant region sequence with Q5 Polymerase (New England Biolabs). Following Sanger sequencing, full-length variable regions were synthesized as gene blocks (Integrated DNA Technologies) and cloned into hIgG1 and hKappa expression vectors by Gibson assembly. Recombinant antibodies were expressed in Expi293 cells following co-transfection of heavy and light chain plasm ids (1:1 ratio) using Expifectamine 293 (Thermo Fisher Scientific). Supernatants were harvested after 5-6 days, purified by affinity chromatography (Protein A Sepharose, GE), and desalted with a PD-10 (Cytiva) column.

Binding analysis via biolayer interferometry: Biolayer interferometry (BLI) was used to quantify the binding capacity of SARS2-38 Fab fragments to trimerized SARS-CoV-2 spike. 10 μg/mL of biotinylated spike was immobilized onto streptavidin biosensors (ForteBio) for 3 min. After a 30 sec wash, the pins were submerged in running buffer (10 mM HEPES, 150 mM NaCl, 3 mM EDTA, 0.05% P20 surfactant, and 1% BSA) containing SARS2-38 Fab ranging from 1 to 1,000 nM, followed by a dissociation step in running buffer alone. The BLI signal was recorded and analyzed using BIAevaluation Software (Biacore).

Cryo-EM sample preparation: Data were collected on lacey carbon grids with or without ultra-thin carbon film. For standard lacey carbon grids (Ted Pella #01895-F), SARS-CoV-2 spike was prepared at 1 mg/mL in TBS (30mM Tris pH 8, 150 mM NaCl). For lacey carbon grids with ultra-thin carbon film (Ted Pella #01824G), SARS-CoV-2 spike was prepared at 0.2 mg/mL in TBS. Each sample was incubated for 15 min with 1 molar equivalent of SARS2-38 Fab fragments, applied to glow-discharged grids, then flash-frozen in liquid ethane using a Vitrobot Mark IV (ThermoFisher Scientific).

Cryo-EM data collection: Grids were loaded into a Cs-corrected FEI Titan Krios 300 kV microscope equipped with a Falcon 4 direct electron detector. Images were collected at a nominal magnification of 59000×, resulting in a pixel size of 1.16 Å. Each movie consisted of 50 frames at 260 ms each with a dose of 1e-/ÅA2/frame, yielding a total dose of 50e-/Å2/movie.

Cryo-EM data processing: Movies were motion corrected using MotionCor2 v1.3.1, and contrast transfer function parameters were estimated using GCTF v1.18. Particles were picked using a general model in CrYOLO v1.7.6. 2D classification was performed in Relion 3.1, and particles in good classes from grids with or without ultra-thin carbon were combined for further processing. These particles were subjected to 3D classification, and those from the best class (all RBDs in the down position, with one bound by Fab) were selected for iterative Bayesian polishing and per-particle CTF refinement in Relion 3.1. These particles were then used in non-uniform refinement in cryoSPARC v3.1.0 to generate a full-spike map. To improve map quality at the Fab/spike interface, a mask was generated encompassing only the Fv and RBD, and particles were subjected to local non-uniform refinement in cryoSPARC v3.1.0. Final maps were sharpened via deep learning employed through DeepEMhancer.

Model building: The locally refined map was used to construct a model of the RBD bound by SARS2-38 Fv. An initial model for the RBD was adapted from a crystal structure of RBD bound to ACE2 (PDB 6M0J). For initial modeling of SARS2-38 Fv, pBLAST was used to identify pre-existing Fab structures with high sequence similarity (PDB 1KIQ for VH, and PDB 5XJM for VL). These starting components were combined and docked into the map, then refined in Coot v0.9.5, Isolde v1.1.0, and Phenix v1.19. Epitope and paratope contacts were identified using qtP ISA and structures were visualized using UCSF ChimeraX.

The full-spike map was used to construct a model of the spike bound by one Fv with all RBDs in the down position. An initial model was generated by combining the locally refined Fv/RBD structure with a previously solved cryo-EM structure of trimeric SARS-CoV-2 spike in the proper RBD configuration (PDB 6VXX). This model was docked into the full-spike map then refined using Coot v0.9.5, Isolde v1.1.0, and Phenix v1.19.

RBD conservation analysis: RBD sequence data (residues 333-520) were retrieved on March 28, 2021 from the COVID-19 CoV Genetics Browser (covidcg.org), enabled by data from GISAID. In total, 786,273 sequences were included in the analysis. Probability of conservation relative to the reference sequence (2019n-CoV/WA1/2020) was computed for each residue, and results were log-transformed and normalized to generate a per-residue conservation score (1=complete conservation, 0=zero conservation). Results were visualized using a color-coded surface rendering of the RBD in UCSF ChimeraX.

Neutralization assays: FRNTs were performed as described previoulsy. Briefly, serial dilutions of antibody were incubated with 2×102 FFU of SARS-CoV-2 for 1 h at 37° C. Immune complexes were added to cell monolayers (Vero E6 cells or other cell lines where indicated) and incubated for 1 h at 37° C. prior to the addition of 1% (w/v) methylcellulose in MEM. Following incubation for 30 h at 37° C., cells were fixed with 4% paraformaldehyde (PFA), permeabilized and stained for infection foci with SARS2-16 (hybridoma supernatant diluted 1:6,000 to a final concentration of ˜20 ng/mL) when using SARS-CoV-2 isolate WA1/2020, or with a mixture of mAbs that bind various epitopes on the RBD and NTD of spike (SARS2-02, SARS2-11, SARS2-31, SARS2-38, SARS2-57, and SARS2-71; diluted to 1 μg/mL total mAb concentration) for the VOCs. Antibody-dose response curves were analyzed using non-linear regression analysis (with a variable slope) (GraphPad Software). The antibody half-maximal inhibitory concentration (EC50) required to reduce infection was determined.

Pre- and post-attachment neutralization assays: For pre-attachment assays, serial dilutions of mAbs were prepared at 4° C. in Dulbecco's modified Eagle medium (DMEM) with 2% FBS and preincubated with 10² FFU of SARS-CoV-2 for 1 h at 4° C. MAb-virus complexes were added to a monolayer of Vero cells for 1 h at 4° C. Virus was allowed to internalize during a 37° C. incubation for 30 min. Cells were overlaid with 1% (wt/vol) methylcellulose in MEM. For post-attachment assays, 2×102 FFU of SARS-CoV-2 was adsorbed onto a monolayer of Vero cells for 1 h at 4° C. After removal of unbound virus, cells were washed twice with cold DMEM, followed by the addition of serial dilutions of MAbs in cold DMEM. Virus-adsorbed cells were incubated with mAd dilutions for 1 h at 4° C. Virus then was allowed to internalize for 30 min at 37° C., and subsequently cells were overlaid with methylcellulose as described above. Thirty hours later, plates were fixed with 4% PFA and analyzed for antigen-specific foci as described above for FRNTs.

Attachment inhibition assay: SARS-COV-2 was incubated with mAbs at 10 μg/mL for 1 h at 4° C. The mixture then was added to pre-chilled Vero E6, Vero-TMPRSS2, Vero-TMPRSS2-ACE2, or Calu-3 cells at an MOI of 0.005 and incubated at 4° C. for 1 h. Cells were washed six times with chilled PBS before addition of lysis buffer and extraction of RNA using MagMax viral RNA isolation kit (Thermo Fisher Scientific) and a Kingfisher Flex 96-well extraction machine (Thermo Fisher Scientific). SARS-CoV-2 RNA was quantified by qRT-PCR using the N-specific primer/probe set described below. GAPDH was measured using a predesigned primer/probe set (IDT PrimeTime Assay Hs.PT.39a.22214836). Viral RNA levels were normalized to GAPDH, and the fold change was compared with isotype control mAb. For each cell type, a control with a 4-fold lower MOI (0.00125) was included to demonstrate detection of decreased viral RNA levels.

Virus internalization assay: SARS-COV-2 was incubated with mAbs at 10 μg/mL for 1 h at 4° C. The mixture was then added to pre-chilled Vero E6 cells at an MOI of 0.005 and incubated at 4° C. for 1 h. Cells were washed twice with chilled PBS to remove unbound virus, and subsequently incubated in DMEM at 37° C. for 30 min to allow virus internalization. Cells then were treated with proteinase K and RNaseA at 37° C. for 10 min to removed uninternalized virus. Viral and cellular RNA were extracted and analyzed as described above for the attachment inhibition assay. A no internalization control was included, where proteinase K and RNase A treatments were performed directly after washing, without an internalization step.

Measurement of viral burden and cytokine and chemokine levels: On 7 dpi, mice were euthanized and organs were collected. Nasal washes were collected in 0.5 mL of PBS. Organs were weighed and homogenized using a MagNA Lyser (Roche). Viral RNA from homogenized organs or nasal wash was isolated using the MagMAX Viral RNA Isolation Kit (ThermoFisher) and measured by TaqMan one-step quantitative reverse-transcription PCR (RT-qPCR) on an ABI 7500 Fast Instrument. Viral burden is expressed on a log10 scale as viral RNA per mg for each organ or total nasal wash after comparison with a standard curve produced using serial 10-fold dilutions of viral RNA standard. For the measurement of cytokine and chemokine levels in the lung, lung homogenates were treated with 1% Triton X-100 for 1 h at room temperature to inactivate virus. Cytokine and chemokine levels in the lung homogenate were then analyzed by multiplex array (Eve Technologies Corporation).

What is claimed is:

1. An isolated antibody or antigen-binding fragment thereof comprising a light chain variable region comprising an L1 of SEQ ID NO: 1, an L2 of DTS, an L3 of SEQ ID NO: 2, or any combination thereof; and/or a heavy chain variable region comprising an H1 of SEQ ID NO: 3, an H2 of SEQ ID NO: 4, an H3 of SEQ ID NO: 5, or any combination thereof.

2. The isolated antibody or antigen-binding fragment of claim 1, wherein the amino acid sequnce of the light chain variable region comprises SEQ ID NO: 6; and/or the amino acid sequence of the heavy chain variable region comprises SEQ ID NO: 7.

3. The antibody or antigen-binding fragment of claim 1, wherein the antibody or antigen-binding fragment specifically binds to a conserved epitope proximal to the receptor biding domain (RBD) of the coronavirus spike protein.

4. The antibody or antigen-binding fragment of claim 3, wherein the epitope comprises amino acids K444 and G446.

5. The antibody or antigen-binding fragment of claim 1, wherein the antibody or antigen-binding fragment neutralizes the coronavirus with an IC₅₀ of about 0.0001 μg/ml to about 30 μg/ml.

6. The antibody or antigen-binding fragment of claim 5, wherein the coronavirus comprises SARS-CoV-2.

7. The antibody or antigen-binding fragment of claim 1, wherein the antibody or antigen-binding fragment is humanized.

8. The antibody or antigen-binding fragment of claim 1, wherein the antibody or antigen-binding fragment is a monoclonal antibody and/or is an IgG antibody.

9. The antibody or antigen bind fragment of claim 1, wherein the antibody or antigen bind fragment comprises a hinge region or Fc region that contains at least one amino acid substitution, deletion, or insertion relative to the sequence of a wild-type hinge region or a wild-type Fc region.

10. The antibody or antigen bind fragment of claim 1, wherein the antibody or antigen binding fragment comprises an Fc region that contains at least one amino acid substitution, deletion, or insertion relative to the sequence of a wild-type Fc region.

11. The antibody or antigen bind fragment of claim 10, wherein the substitution, deletion, or insertion prevents or reduces recycling of the antibody or antigen-binding fragment.

12. A pharmaceutical composition comprising an antibody of claim 1 and a pharmaceutically acceptable carrier or excipient.

13. The pharmaceutical composition of claim 12, further comprising a dispersing agent, buffer, surfactant, preservative, solubilizing agent, isotonicity agent, or stabilizing agent.

14. The pharmaceutical composition of claim 13, wherein said carrier comprises physiological saline, ion exchanger, alumina, aluminum stearate, lecithin, serum protein, human serum albumin, buffer, phosphate, glycine, sorbic acid, potassium sorbate, partial glyceride mixture of saturated vegetable fatty acids, water, salts or electrolytes, protamine sulfate, disodium hydrogen phosphate, potassium hydrogen phosphate, sodium chloride, zinc salt, colloidal silica, magnesium trisilicate, polyvinyl pyrrolidone, cellulose, polyethylene glycol, sodium carboxymethylcellulose, polyacrylates, wax, polyethylene-polyoxypropylene-block polymer, polyethylene glycol, wool fat, or a combination thereof.

15. A method of preventing or treating a coronavirus infection in a subject in need thereof, the method comprising administering to the subject a therapeutically effective amount of a compostion comprising an antibody or antigen-binding fragment, wherein the antibody or antigen-binding fragment comprises a light chain variable region comprising an L1 of SEQ ID NO: 1, an L2 of DTS, an L3 of SEQ ID NO: 2, or any combination thereof; and/or a heavy chain variable region comprising an H1 of SEQ ID NO: 3, an H2 of SEQ ID NO: 4, an H3 of SEQ ID NO: 5, or any combination thereof.

16. The method of claim 15, wherein the composition is administered intramuscularly, intravenously, intradermally, or intranasally.

17. The method of claim 15, wherein the composition is administered therapeutically to treat an active coronavirus infection.

18. The method of claim 15, wherein the composition is administered prophylactically to prevent a coronavirus infection.

19. The method of claim 15, wherein the coronavirus infection is COVID-19.

20. The method of claim 15, further comprising administering an antiviral drug selected from baloxavir, oseltamivir, zanamivir, peramivir or any combination thereof.


SEQUENCE LISTING (17 sequences)

SEQ ID NO: 1 - protein, 5 aa, Artificial Sequence
  Synthesized
STVSF

SEQ ID NO: 2 - protein, 9 aa, Artificial Sequence
  Synthesized
QQWNTYPLT

SEQ ID NO: 3 - protein, 8 aa, Artificial Sequence
  Synthesized
GFSLTRYG

SEQ ID NO: 4 - protein, 7 aa, Artificial Sequence
  Synthesized
IWADGST

SEQ ID NO: 5 - protein, 10 aa, Artificial Sequence
  Synthesized
ARDGRGYDDY

SEQ ID NO: 6 - protein, 106 aa, Artificial Sequence
  Synthesized
QIVLTQSPAIMSASPGEKVTMTCSASSTVSFIYWYQQKPGSSPRLLIYDTSNPASGVPVRFSGSGCGTSYYLTISRMEAEDAATYYCQQWNTYPLTFGAGTKLELK

SEQ ID NO: 7 - protein, 186 aa, Artificial Sequence
  Synthesized
TNLCPFGEVFNATRFASVYAWNRKRISNCVADYSVLYNSASVSTFKCYGVSPTKLNDLCFTNVYADSFVIRGDEVRQIAPGQTGKIADYNYKLPDFTGCVIAWNSNNLDSKVGGNYNYLYRLFRKSNLKPFERDISTEIYQASGSTPCNGVEGFNCYFPLQSYGFQPTNGVGYQPYRVVVLSFELL

SEQ ID NO: 8 - protein, 186 aa, Artificial Sequence; identical to SEQ ID NO: 7
  Synthesized

SEQ ID NO: 9 - protein, 186 aa, Artificial Sequence
  Synthesized
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELL

SEQ ID NO: 10 - protein, 186 aa, Artificial Sequence
  Synthesized
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELL

SEQ ID NO: 11 - protein, 186 aa, Artificial Sequence
  Synthesized
TNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELL

SEQ ID NO: 12 - protein, 186 aa, Artificial Sequence
  Synthesized
TNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELL

SEQ ID NO: 13 - protein, 186 aa, Artificial Sequence
  Synthesized
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLFRLFRKSNLKPFERDISTEIYQAGSTPCNGVEGFNCYFPLQSYGFQPTNGVGYQPYRVVVLSFELL

SEQ ID NO: 14 - protein, 186 aa, Artificial Sequence
  Synthesized
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRFRLFRKSNLKPFERDISTEIYQAGSTPCNGVEGFNCYFPLQSYGFQPTNGVGYQPYRVVVLSFELL

SEQ ID NO: 15 - protein, 186 aa, Artificial Sequence
  Synthesized
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSKNLDSKVGGNYNYLFRLFRKSNLKPFERDISTEIYQAGSTPCNGVEGFNCYFPLQSYGFQPTNGVGYQPYRVVVLSFELL

SEQ ID NO: 16 - protein, 186 aa, Artificial Sequence
  Synthesized
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPDCTPPALNCYWPLNDYGFYTTTGIGYQPYRVVVLSFELL

SEQ ID NO: 17 - protein, 194 aa, Artificial Sequence
  Synthesized
GVECDFSPLLSGTPEQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLPLEGGGWLVASGSTVAMTEQLQMGFGIT